(19) United States
(12) Patent Application Publication
(10) Pub. No.: US 2022/0098547 A1
(43) Pub. Date: Mar. 31, 2022

(54) HYDROGEL BIOMIMETIC FOR INVASIVE DISEASES

(71) Applicants: Molly Sandra SHOICHET, Toronto, Ontario (CA); William Lloyd STANFORD, Ottawa, Ontario (CA); Roger Yue Ting TAM, Ottawa, Ontario (CA); Julien YOCKELL-LELIEVRE, Gatineau, Quebec (CA); Laura Jean SMITH, Toronto, Ontario (CA)

(72) Inventors: Molly Sandra SHOICHET, Toronto, Ontario (CA); William Lloyd STANFORD, Ottawa, Ontario (CA); Roger Yue Ting TAM, Ottawa, Ontario (CA); Julien YOCKELL-LELIEVRE, Gatineau, Quebec (CA); Laura Jean SMITH, Toronto, Ontario (CA)

(21) Appl. No.: 17/298,352

(22) PCT Filed: Nov. 29, 2018
(86) PCT No.: PCT/CA2019/051706

§ 371 Date: May 28, 2021

Related U.S. Application Data
(60) Provisional application No. 62/772,807, filed on Nov. 29, 2018.

(51) Int. Cl. C12N 5/00; G01N 33/50

(52) U.S. Cl. CPC C12N 5/0068; G01N 33/5029; C12N 2537/10; C12N 2533/80; C12N 2533/52

(57) ABSTRACT

An extracellular biomimetic for assessing and analyzing cell invasion includes hydrogel matrix and a first peptide crosslinked to the hydrogel matrix, where the first peptide is responsive to a first substance released by diseased cells upon invasion into the biomimetic. The biomimetic further includes at least one modulating agent enabling cell invasion independent from said first substance. The hydrogel matrix can comprise hyaluronate modified with furanyl functional groups, and the modulating agent can be viscoelastic polymer forming reversible crosslinks within the hydrogel matrix. Examples of the viscoelastic polymer include methyl cellulose, or functionalized methyl cellulose, for example, with thiol functional groups. The first substance released by diseased cells is an enzyme, for example, matrix metalloproteinase (MMP). The biomimetic can be used for drug screening to identify compounds that reduce the invasion and viability of the diseased cells, for example, cells from the lung, brain, breast, prostate, and human pluripotent stem cells.

### FIELD

The present disclosure relates to a 3-D hydrogel biomimetic for assessing and analyzing invasive diseases. The present disclosure also relates to a drug screen method based on the 3-D hydrogel biomimetic.

### BACKGROUND

Cell invasion is a critical hallmark of metastatic diseases.^{[1]} There are limited drug therapies that can effectively inhibit both cell invasion and viability of diseased, invasive cells, and when drugs target only one, it can be devastating for the patient. For example, it has been reported that some glioblastoma patients treated with the anti-VEGF-A monoclonal antibody, bevacizumab, showed increased tumor metastasis despite decreased tumor sizes.^{[2]} Therefore, drug screening methods that can accurately assess both of these functions are crucial in discovering anti-metastatic therapies, yet such screens are lacking. In vitro methods such as transwell plates and Boyden chambers are well established to study cell invasion in response to drug treatments, but these non-physiological platforms do not provide a biomimetic microenvironment to adequately model cell-matrix interactions involved in the complex mechanisms of cell invasion; they provide detailed information about neither viability nor invasiveness of individual cells that is necessary to dissect the therapeutic potential of anti-metastatic drugs. Moreover, they are incompatible with high throughput screening (HTS).

Cell culture using biomimetic 3D hydrogels is an effective strategy to provide cells with the necessary physical and chemical stimuli to promote native cell growth and function.^{[3]} Compared to 2D tissue culture on plastic or glass, 3D hydrogels can be remodeled by cells to permit their invasion into the gels. While natural 3D scaffolds (e.g. decellularized extracellular matrix (ECM),^{[4]} collagen l,^{[1a]} Matrigel^{[5]}) have been used to study cell invasion, their physicochemical properties cannot be readily or independently modified to model the ECM of specific diseases. Conversely, synthetic materials can be tuned to mimic the native microenvironment,^{[6]} but these can be overly simplistic to accurately model native cellular functions. To model cell invasion, protease-degradable synthetic gels have been designed; ^{[7]} however, there are very few gels that permit cells to invade by protease-independent mechanisms.^{[8]} It is key to model cell invasion by both mechanisms in drug screening of metastatic diseases because clinical trials involving matrix metalloproteinase (MMP) inhibitors alone have historically failed.^{[9]} Notwithstanding the advantages of using 3D biomaterials to study cell invasion, their application in HTS to identify drugs that inhibit both cellular invasion and viability has been limited,^{[10]} as most are unsuitable for moderate- to high-throughput screening.

Accurately quantifying both invasion and viability of individual cells remains a challenge in larger drug screens, but is critical because the increased invasiveness of a few robust surviving cells can be devastating to disease progression, yet difficult to distinguish using assays that rely on homogenous fluorescence detection. The discovery of drugs that inhibit cell invasion is further complicated by the lack of 3D hydrogel platforms that both model the complex mechanisms of cell invasion that occur in cancer metastasis^{[1]} and discern differences between healthy versus cancer cells.

### SUMMARY

The present disclosure provides an extracellular biomimetic for culturing diseased cells, comprising:

hydrogel matrix,

a first extracellular matrix protein-mimetic peptide crosslinked to the hydrogel matrix, the first extracellular matrix protein-mimetic peptide being responsive to a first substance released by diseased cells upon invasion into the extracellular biomimetic, and

at least one modulating agent enabling cell invasion independent from the first substance.

The hydrogel matrix may comprise hyaluronate or hyaluronic acid, modified with furanyl functional groups. The furanyl functional groups may be furan, or furan substituted with alkyl-, aryl-, or electron-donating functional groups.

The modulating agent may be at least one viscoelastic component, forming reversible crosslinks within the hydrogel matrix.

The viscoelastic component may be a peptide or protein which may comprise methyl cellulose, alginate crosslinked with calcium cations, amphiphilic block polymers, amphiphilic block polypeptides, coiled-coil peptides, reconstituted basement membrane protein extract, laminin, or collagen.

The viscoelastic polymer may comprise methyl cellulose having thiol functional groups. The viscoelastic polymer, peptide or protein may be modified to comprise any one of aldehyde, ketone, hydrazine functional groups.

The first extracellular matrix protein-mimetic peptide may be further immobilized to the viscoelastic polymer.

The extracellular biomimetic may further comprise a second extracellular matrix protein-mimetic peptide immobilized to the hydrogel matrix and/or the viscoelastic polymer. The second peptide may be present in the amount of less than 1000 μM.

This second extracellular matrix protein-mimetic peptide may be present in the amount of 25 μM to 250 μM.

This second extracellular matrix protein-mimetic peptide may be any one or combination of vitronectin-mimetic peptide and fibronectin-mimetic peptide.

The first substance released by diseased cells may be an enzyme. This enzyme may be matrix metalloproteinase (MMP).

The first extracellular matrix protein-mimetic peptide may be maleimide-modified collagen I-derived peptide crosslinker degradable by the MMP.

The present disclosure also provides a cell culture kit comprising the above mentioned extracellular biomimetic and diseased cells. These diseased cells are from any invading cells, such as any one of the lung, brain, breast, prostate, skin, liver, colon, pancreas, thyroid, bone, muscle, human pluripotent stem cells and their subsequently differentiated cells.

The diseased cells comprise cells isolated from lung cancer patients or derived from human pluripotent stem cells (hiPSCs) to model lymphangioleiomyomatosis (LAM).

The diseased cells may comprise hiPSC-derived smooth muscle cells (SMCs) that model lymphangioleiomyomatosis (LAM-SMCs).

The diseased cells may comprise cells isolated or derived from brain cancer patients to model glioblastoma (GBM).

The diseased cells may comprise cells treated with one or any combination of inhibitors selected from the group consisting of those that inhibit:
ABL1
ADENOSINE DEAMINASE
AKT3
ALK
ANDROGEN
AROMATASE
AURORA KINASE
BCL-2
BRAF
BRD
BTK
CALCINEURIN
CCR5
CDK
CXCR
CYTOCHROME P450
DAGK
DNA METHYLTRANSFERASE
DNA TOPOISOMERASE
EGFR
EPH
ERK
Fibroblast Growth Factor Receptors
FARNESYLTRANSFERASE
FLT
FRAP
GSK3
HDAC
HEAT SHOCK PROTEIN
HEDGEHOG
ITGB1
IRE1
JAK2
KDR
KINESIN-LIKE SPINDLE PROTEIN
KIT
LCK
LIMK1
LYN
MAP2K
MDM2
P38B
P70S6K
PARP
PDGFR
PI3K
PKC
PLK1
PIM2
PROTEASOME
RAF1
Rho-associated protein kinase
RET
Src
SIRT2
SPHINGOSINE KINASE
TANKYRASE
TUBULIN
WNT,
   or one or any combination of agonists selected from the group consisting of:
GLUCOCORTICOID
PKM2
PROGESTERONE
RXR
S1P RECEPTOR.

The cell culture kit may have 6, 24, 48, 96, 384 or 1536 well plates.

The present disclosure provides a drug screening method comprising:
culturing diseased cells in an extracellular biomimetic, the extracellular biomimetic comprising:
a hydrogel matrix,
a first extracellular matrix protein-mimetic peptide crosslinked to the hydrogel matrix, the first extracellular matrix protein-mimetic peptide being responsive to a first substance released by diseased cells upon invasion into the extracellular biomimetic, and
at least one modulating agent enabling cell invasion independent from the first substance;
quantifying invasion and viability of the diseased cells;
administering candidate drug compounds to the biomimetic; and
identifying compounds that reduce both the invasion and viability of the diseased cells.

The quantifying step may comprise measuring the invasion of the diseased cells by staining cells with fluorescent dyes, automated confocal imaging, and automated analysis by an image analysis software program such as custom Image J macros.

The quantifying step may comprise measuring the viability of the diseased cells by staining the dead cells with fluorescent dyes, automated microscopic imaging such as confocal imaging, and automated analysis by an image analysis software program such as custom Image J macros.

Each plate of the cell culture kit may contain both diseased cells and control cells.

A further understanding of the functional and advantageous aspects of the present disclosure can be realized by reference to the following detailed description and drawings.

### BRIEF DESCRIPTION OF THE DRAWINGS

Embodiments will now be described, by way of example only, with reference to the drawings, in which:

FIGS. 1A to 1K illustrate TSC2^{+/-} LAM-SMCs invasion of 3D MMP-degradable HA hydrogels gels via MMP-dependent and -independent mechanisms, where:

FIG. 1A is a schematic representation of potential cell invasion mechanisms.

FIG. 1B is a schematic representation of the composition of biomimetic stimuli-responsive 3D HA hydrogels.

FIG. 1C is a synthetic scheme describing the synthesis of HA-furanyl/MC-SH hydrogels crosslinked with MMP-degradable peptides, and immobilized with cell-adhesive peptides.

FIG. 1D shows Stress relaxation of HA-furanyl/MMP hydrogels is increased with thiolated methylcellulose (MC-SH, solid line) compared to gels without MC-SH (dashed line)

FIG. 1E shows Compressive Modulus of hydrogels with or without 0.5 mg/mL MC-SH are not statistically different. N=4. Mean+SD.

FIG. 1F shows TSC2^{+/-} LAM-smooth muscle cells (LAM-SMCs) isolated from patient-derived iPSCs cultured on biomimetic 3D hyaluronan (HA)-based hydrogels are more invasive than

FIG. 1G shows iPSC-derived TSC2^{+/+} control SMCs.

FIG. 1H shows MMP-2 and MMP-9 expression in invasive TSC2^{+/-} LAM-SMCs (black bars) relative to TSC2^{+/+} cells (dashed line) and TSC2^{-/-} angiomyolipoma cells (stripe lined bars), assessed by zymography of conditioned media isolated from the various cell types. N=3, **p<0.01 represents a significant difference from TSC2^{+/+} control SMCs and TSC2^{-/-} angiomyolipoma cells for MMP9, and a significant difference between TSC2^{+/-} LAM-SMCs and TSC2^{-/-} angiomyolipoma cells for MMP2.

FIG. 1I shows GM6001 partially inhibits invasion of TSC2^{+/-} LAM-SMCs into 3D hydrogels. N=3, *p<0.05.

FIG. 1J shows Hydrogels lacking thiolated methylcellulose (MC-SH) with decreased invasion of each cell type. N=3.

FIG. 1K shows Treatment with saracatinib or Y-27632 decrease cell invasion. N=4. For J, K:. *p<0.05, **p<0.01, ***p<0.001.

FIGS. 2A to 2I illustrate Patient-derived (TSC2^{+/-}) LAM-SMCs exhibiting LAM-like characteristics when cultured in 3D HA hydrogels, where:

FIG. 2A is a schematic representation showing cells cultured on 2D polystyrene vs. 3D hydrogels.

FIG. 2B shows Patient-derived (TSC2^{+/-}) LAM-SMCs express lower levels of TSC2 when cultured on 3D HA gels vs. on 2D polystyrene. Conversely, TSC2^{+/+} control SMCs express higher levels of TSC2 when cultured on 3D HA gels vs. 2D polystyrene. N=4. *p<0.05, **p<0.01 indicates significant differences between each respective cell type cultured on 3D vs. 2D (horizontal line). ***p<0.001 indicates significant difference between TSC2^{+/+} control SMCs and TSC2^{+/-} LAM-SMCs cultured on 3D gels.

FIG. 2C is a schematic representation depicting the culture of TSC2^{+/-} LAM-SMCs vs. TSC2^{+/+} control SMCs on 3D HA hydrogels.

FIG. 2D and FIG. 2E show representative confocal images showing the expression of CD44v6 . Cells are stained with Hoechst (for nuclei) and anti-CD44v6 (for CD44v6). Scale bar represents 100 μm.

FIG. 2F shows Patient-derived TSC2^{+/-} LAM-SMCs cultured on 3D HA hydrogels express higher levels of CD44v6 vs. TSC2^{+/+} control SMCs.

FIG. 2G is a schematic and quantification of CD44v6 expression of invasive (>100 μm) vs non-invasive (<100 μm) cells. For F, G: N=3, *p<0.05, ***p<0.001.

FIG. 2H and FIG. 2I show representative confocal images demonstrate expression of the hyaluronan receptor CD44 on both TSC2^{+/-} LAM-SMCs (FIG. 2H) and TSC2^{+/+} control SMCs FIG. 2I).

FIGS. 3A to 3G illustrate 3D biomimetic in vitro model of LAM and application to automated analysis of cell invasion and viability, where:

FIG. 3A shows TSC2^{+/-} LAM-smooth muscle cells (LAM-SMCs) isolated from patient-derived iPSCs cultured on biomimetic 3D hyaluronan (HA)-based hydrogels enable cells to recapitulate their native growth compared to conventional culture on 2D tissue culture polystyrene. 3D cell culture allows drugs to be screened for cell viability and invasion (B-D) Response of LAM-SMCs and control SMCs treated with 20 nM rapamycin (mTORC1 inhibitor), the only clinically approved therapy for LAM.

FIG. 3B shows viability of cells cultured on 2D and 3D HA gels, normalized to cells cultured on the same substrate treated with DMSO (dotted line).

FIG. 3C shows cell invasion into 3D HA gels. (N=4, **p<0.01)

FIG. 3D shows active MMP-9 expression, assessed by zymography of conditioned media. (N=3, *p<0.05).

FIG. 3E is a Schematic representation of algorithm used to automatically quantify cell invasion using an Image J macro. The gel surface is demarcated with silica particles, and Z-stack images are captured for each channel; the XYZ coordinate of each cell is identified, and at each cellular XY coordinate, the distance of cell invasion is equal to the difference between the maximum signal along the Z-axis of the gel surface marker (dashed line curve) and the cell (solid line curve).

FIG. 3F and FIG. 3G are heat maps showing the inhibition of FIG. 3F average cell invasion, and FIG. 3G cell viability of LAM-SMCs versus control SMCs treated with 80 kinase inhibitor drugs (at 5 μM). Lighter shades of gray within the heatmap indicate greater selectivity and efficacy in terms of reduced invasion and viability of LAM-SMCs versus control SMCs, while darker coloured boxes indicate the opposite (and undesirable) drug response. The order of drug names in FIG. 3F corresponds to their respective target pathway listed in FIG. 3G. Columns represent biological replicates.

FIGS. 4A to 4R illustrates lung cancer cells expressing CD44 and showing varying invasiveness into 3D HA hydrogels, where:

FIG. 4A to FIG. 4I show Primary cells isolated and cultured from three separate lung carcinoma biopsies identified as adenocarcinoma (FIG. 4A to FIG. 4C), squamous cell carcinoma (FIG. 4D to FIG. 4F), and neuroendocrine tumor (FIG. 4G to FIG. 4I).

FIG. 4J and FIG. 4L show non-small cell lung cancer (NCI-H1299) and

FIG. 4M to FIG. 4O show small cell lung cancer (NCI-H446) cells.

FIG. 4P to FIG. 4R show that healthy human bronchial epithelial control cells do not invade into 3D hydrogels. FIG. 4A, 4D, 4G, 4J, 4M show that Lung cancer cells express CD44, while (P) healthy bronchial epithelial cells do not.

FIG. 5 shows ¹H NMR of hyaluronan-furanyl in D₂O, 500 MHz, 256 scans: with 65% furanyl substitution. Peaks at 7.5 and 6.4 ppm correspond to the three aromatic furanyl protons, and 2.0 ppm corresponds to the —CH₃ of N-acetyl group. Quantification of furanyl substitution is calculated by comparing the integration of the furanyl and methyl protons.

FIG. 6 shows Compressive Modulus of rat lung tissue and 3D HA hydrogels within the range of normal healthy human lung tissue. N=4. Mean+standard deviation. Unpaired two-tailed t-test. *p<0.05.

FIG. 7 shows that active MMPs degrade gelatin/polyacrylamide gels (white bands). Media used in assays contain 1% FBS, which contains low concentrations of MMP2, thereby accounting for the faint band observed in media controls.

FIG. 8 shows the effect of Src and ROCK inhibition on MMP expression and cell viability. Response of patient TSC2^{+/-} LAM-SMCs and TSC2^{+/+} control SMCs to treatment with (A,B) saracatinib (Src inhibitor, 0.5 82 M), and (C,D) Y-27632 (ROCK inhibitor, 10 μM). (A,C) Active MMP-9 expression, assessed by zymography of conditioned media. N=3 biological replicates, **p<0.01 indicates significant difference between MMP-9 expression of TSC2^{+/-} LAM-SMCs vs TSC2^{+/+} control SMCs. One-way ANOVA, Tukey's post-hoc test. (B, D) Viability of cells cultured on 3D HA gels, normalized to DMSO vehicle control. N=4 biological replicates; mean+standard deviation, no statistical significances between drug treatments vs DMSO vehicle controls of each respective cell type.

FIG. 9 shows the expression of integrins αV, β1, β3, are increased in iPSC-derived TSC2^{+/-} LAM-SMCs. Quantification and representative confocal images showing immunofluorescence of various integrin subunits in patient-derived TSC2^{+/-} LAM-SMCs (black bars) and TSC2^{+/+} control SMCs (dotted bars). Anti-integrin (A-C) αV; (D-F) β1; (G-I) β3. Nuclei are stained with Hoechst. Scale bar=100 μm. N=3 biological replicates, bars show mean+standard deviation, unpaired two-tailed t-test *p<0.05, **p<0.01.

FIG. 10 shows the growth and invasion of LAM-SMCs are dependent on vitronectin concentration. (A) Schematic representation showing the effect of vitronectin peptide concentration on the growth and invasion of LAM-SMCs. (B-E) Quantification of (B) percentage and (C) number of invasive cells, and cell numbers for (D) TSC2^{+/-} LAM-SMCs and (E) TSC2^{+/+} control SMCs in HA gels with varying vitronectin concentrations. N≥3 biological replicates. Mean+standard deviation. For B,C: Two-way ANOVA, Tukey post-hoc test. *p<0.05, **p<0.01, ***p<0.001, ****p<0.0001, n.s.=not significant. For D,E: One-way ANOVA, Uncorrected Fisher's LSD test. *p<0.05. (F-M) Representative confocal images showing cell growth of (F-I) TSC2^{+/-} LAM-SMCs, and (J-M) TSC2^{+/+} control SMCs in HA gels with varying vitronectin concentrations. Cell nuclei are stained with Hoechst. White dashed line is shown to demarcate the same depth in each gel. Scale bar=100 μm.

FIG. 11 shows the effect of vitronectin concentration on cell viability and proliferation. Quantification of cell viability and proliferation of (A,C,E,G) LAM patient-derived TSC2^{+/-} and (B,D,F,H) control TSC2^{+/+} SMCs in hydrogels with varying concentrations of vitronectin-mimetic peptide (0, 25, 250, 1000 μM). (A,B) Percentage of viable cells, as assessed by calcein AM staining. (C,D) Percentage of proliferating (Ki67⁺) cells. (E-H) Mean fluorescence intensity of (E,F) apoptotic (Annexin V⁺) and (G,H) necrotic (Propidium iodide⁺) cells. Mean+standard deviation. N=3 biological replicates. *p<0.05, **p<0.01, ****p<0.0001. One-way ANOVA, Tukey post-hoc analysis.

FIG. 12 shows Cell invasion of TSC2^{+/-} LAM patient-derived SMCs and TSC2^{+/+} control SMCs cultured on optimized HA hydrogels used in this study, and commercially-available Collagen I hydrogels. There is a greater difference between the two cell types cultured in optimized HA hydrogels compared to collagen I gels. N=3 biological replicates. Mean+standard deviation. Two-way ANOVA, Tukey post-hoc test. **p<0.01.

FIG. 13(A) shows a confocal image of TSC2^{-/-} angiomyolipoma cells cultured on 3D HA hydrogels.

FIG. 13(B) shows the quantification of cell invasion of TSC2^{+/-} LAM-SMCs (black bar), TSC2^{+/+} control SMCs (dotted bar), and TSC2^{-/-} Angiomyolipoma cells (stripe lined bar). N=4 biological repeats, mean+standard deviation. One-way ANOVA, Tukey post-hoc. **p<0.01 represents significant difference of TSC2^{+/-} LAM-SMCs from healthy control TSC2^{+/+} SMCs and TSC2^{-/-} angiomyolipoma cells.

FIG. 14 shows representative scatter plots showing the depth of invasion and viability of (A-C) TSC2^{+/-} LAM SMCs and (D-F) TSC2^{+/+} control SMCs. Cellular invasion and viability is differentially affected in response to treatment with (A,D) DMSO control, (B,E) verteporfin (positive control for dead cells), and (C,F) Y-27632 (positive control for inhibition of cell invasion). Each data point (circle) represents an individual cell, and is quantified using an automated macro on ImageJ. Invasion distance is measured as the difference between the cellular z-axis position and the z-axis position of the gel surface at the same XY position. Cell viability is assessed by treatment with SyTox stain (which stains dead cells). Gray lines within the plots indicate the same threshold set for cell invasion (vertical gray line), and viability (horizontal gray line) relative to the DMSO control for TSC2^{+/+} control SMCs.

FIG. 15 shows the secondary antibody control for CD44 staining. NCI-H446 (small cell lung cancer) cells were stained with (A) donkey anti-mouse secondary antibody-555 alone (i.e. without anti-CD44 antibody). To show overall cell morphology, cells were stained with (B) phalloidin-AlexaFluor488 (for actin); (C) Channel showing Hoechst staining (for nucleus) only. (D) Overlay of all 3 channels showing the absence of secondary Ab-555 signal in the absence of mouse anti-CD44 antibody.

FIG. 16 shows reconstructed confocal z-stack images of human fetal (HF) NSC's (FIG. 16A), and two GBM patient lines grown on top of 3D hydrogels counterstained with hoechst and phalloidin (FIG. 16B and FIG. 16C). GBM patient lines exhibit invasive behaviours into the hydrogels while the HF line remains as a monolayer on top of the gel.

FIG. 17 shows attenuated diptheria toxin-(aDT)-siRNA downregulates ITGB1 expression in glioblastoma stem cells (GSCs) and reduces cellular invasion, in which

FIG. 17A shows aDT-ITGB1 (black striped bars) downregulates ITGB1 mRNA expression compared to negative controls: aDT conjugated to a non-targeting siRNA (aDT-NT, black bars) and ITGB1 siRNA only without lipofectamine (grey checkered bar) at 24 h post treatment. Positive control is transfected ITGB1-siRNA with lipofectamine (solid grey bar). Data is shown as n=3, mean±SD, normalized to an untreated control. Data was analyzed using one-way-ANOVA followed by Tukey's correction on the logarithmic data (* p<0.05, ** p<0.01.

FIG. 17B shows that aDT-ITGB1 reduces invasion compared to controls (no treatment and aDT-NT) in a 3D hydrogel model. Representative images shown. 15 μm beads label the top of the hydrogel; cell nuclei are labeled using Hoechst. All scale bars are 150 μm.

FIG. 17C shows invasion depth was quantified as a percentage of the untreated control. Data was analyzed using one-way-ANOVA followed by Tukey's correction (* p<0.05, ** p<0.01).

FIG. 17D shows that aDT-ITGB1 did not reduce number of adhered cells in a 3D hydrogel model. Representative images are shown. All scale bars are 150 μm.

FIG. 17E shows quantification of the number of adherent cells by counting number of cell nuclei; no significant difference was observed, demonstrating that differences in invasion were due to ITGB1 downregulation. Data was analyzed using one-way-ANOVA followed by Tukey's correction.

### DETAILED DESCRIPTION

Various embodiments and aspects of the disclosure will be described with reference to details discussed below. The following description and drawings are illustrative of the disclosure and are not to be construed as limiting the disclosure. The Figures are not to scale. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. In one non-limiting example, the terms “about” and “approximately” mean plus or minus 10 percent or less.

As used herein, the term “a biomimetic” refers to an extracellular matrix platform that mimics the native microenvironment to model cell-matrix interactions.

As used herein, the term “a diseased cell” refers to a cell that is derived from the tissue or a human pluripotent stem cell that models any invasive disease, such as those of the immune system, lung, skin, brain, breast, prostate, liver, colon, pancreas, thyroid, bone, muscle, lymph, head or neck. The biomimetic platform according to the present disclosure can be used for cancer cells, or other invasive cells types in other diseases beyond cancer cells.

As used herein, the term “an adhesive peptide” refers to amino acid sequences that bind to cell-surface receptors, and are derived from extracellular matrix proteins, for example, vitronectin, fibronectin, laminin, collagen, VCAM, ICAM, NCAM.

As used herein, the term “a modulating agent” refers to any substance, structure or process that modifies the 3-D hydrogel matrix platform to permit cell invasion by a mechanism independent from a well-known effector of a disease, for example, an enzyme released by a diseased cell.

In one example according to an embodiment of the present disclosure, the modulating agent may include one or a combination of a substance, structure and process enabling cell invasion independent from the enzyme matrix metalloproteinase (MMP), secreted by the cells of a patient.

Other examples of modulating agent include at least one viscoelastic polymer forming reversible crosslinks within the hydrogel matrix. Another example is alginate crosslinked with calcium cations.

Unless defined otherwise, all technical and scientific terms used herein are intended to have the same meaning as commonly understood to one of ordinary skill in the art.

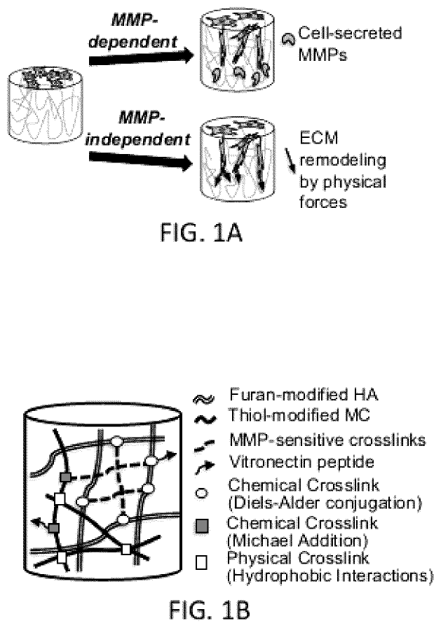

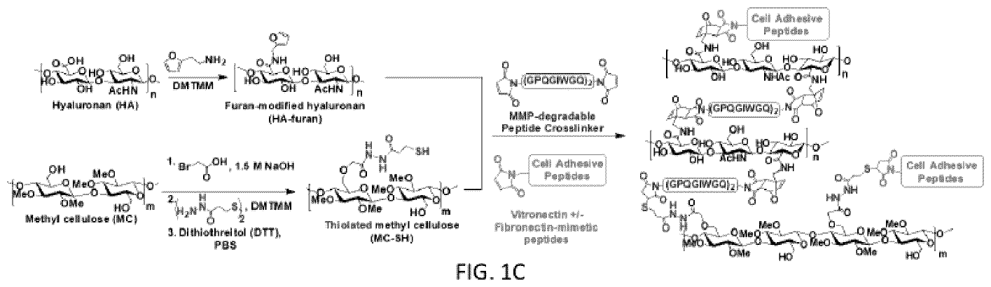

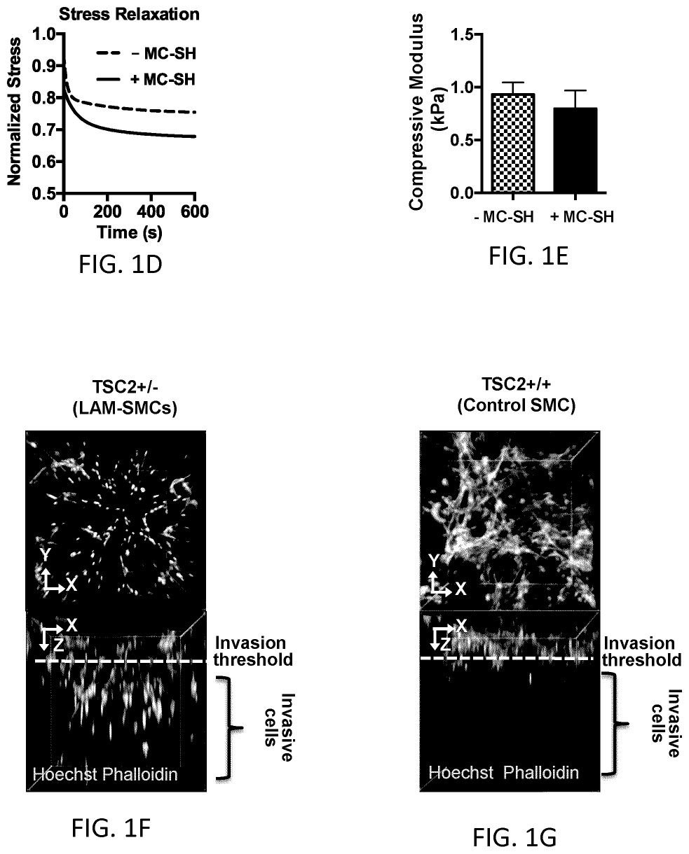

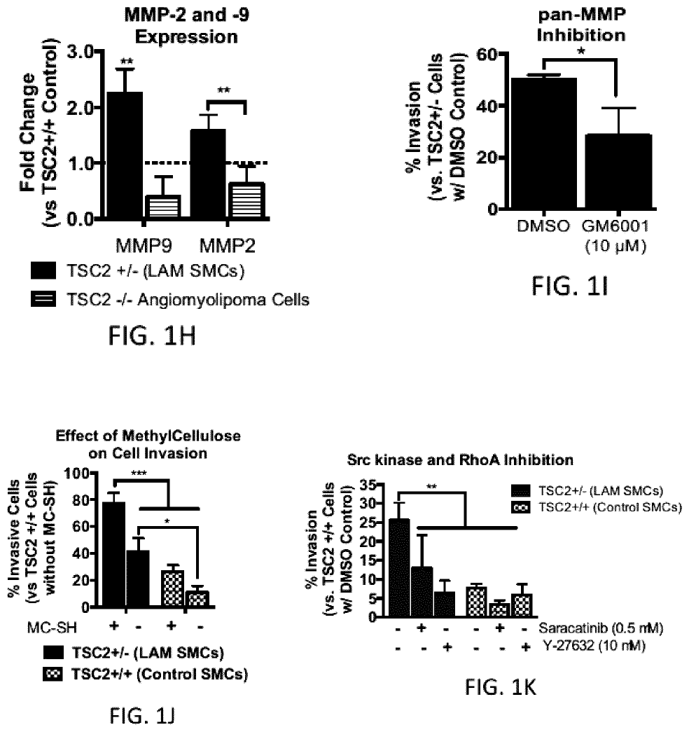

Cell behavior is highly dependent upon microenvironment. Thus, to identify drugs targeting invasive cells, such as cancer cells, of which metastatic cells are an example, screens need to be performed in tissue mimetic substrates that allow cell invasion and matrix remodeling. Recognizing the critical element and complexity of cell invasion to disease progression and therapeutic intervention, the present inventors developed a novel high content, 3D biomimetic hydrogel drug screening platform that permits cell invasion by multiple mechanisms (FIG. 1A), and can independently quantify cell viability and invasion at the individual cell level. The present inventors demonstrate its utility in screening for compounds that can inhibit these functions in a metastatic and destructive cell model, for example, lung disease model, lymphangioleiomyomatosis (LAM), a rare lung neoplasm characterized by: loss of function mutations in TSC1 or TSC2, the expression of smooth muscle cell (SMC) and neural crest markers, hyperactive mTORC1 signaling, and secretion of proteases that remodel and destroy the lung parenchyma.^{[11]} As primary LAM cells do not proliferate in culture, the present inventors have modeled LAM using TSC2 and TSC1 mutant pluripotent stem cell-derived smooth muscle cells (LAM-SMCs) and neural crest cells, which demonstrate hyperactive mTORC1 signaling and express most LAM cell markers.^{[12]} The present inventors also demonstrate its utility in screening for compounds that can inhibit the invasion and viability in a diseased brain cell model, for example, glioblastomas (also called GBM), a common malignant brain tumor. The 3D hydrogel platform according to the present disclosure is rationally-designed to reflect the ECM of the diseased cell and the complex mechanisms of cell invasion, differentiating it from conventional 2D culture on stiff TCPS (which lacks the ability to be remodeled by invasive cells) and 3D culture using natural biomaterials (which cannot have their physical and chemical properties independently modified). In one embodiment of the present disclosure, the hydrogel is composed of hyaluronic acid (HA), which is over-expressed in many invasive cancers,^{[13]} and binds to the cancer-associated cell surface receptor CD44v6 that is upregulated in LAM^{[14]} and various cancer cells.^{[15]}

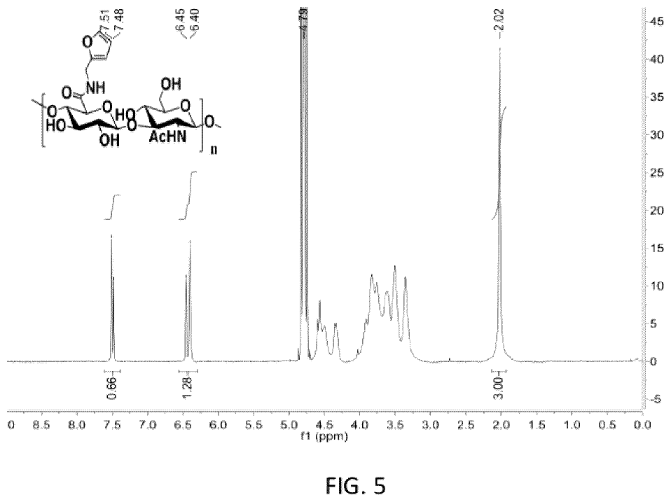

To form stable HA hydrogels that can withstand the duration of drug screen, the HA polymer backbone is modified with furanyl motifs (FIG. 1B) that can form stable, covalent chemical bonds with bismaleimide-terminated peptides via Diels-Alder click chemistry (FIG. 1C).^{[16]} Furanyl motifs include furans comprising alkyl, aryl, or electron-donating substituents. The degree of furanyl modification on HA was quantified by ¹H NMR (FIG. 5), and its presence enables the concentration and composition of maleimide-modified crosslinking and pendant peptides to be customized, which is useful to alter the physical and chemical properties of the hydrogel. Pendant peptides may be extracellular matrix protein-mimetic peptides such as vitronectin- or fibronectin-mimetic peptides.

For the purpose of illustrating exemplary embodiments, the following discussion is provided with reference to diseased lung and brain cells. However, the present disclosure can also be implemented with other cells, including immune and inflammatory cells and diseased cells from the breast, brain, skin, prostate, liver, colon, pancreas, thyroid, bone, muscle, lymph, ovarian, cervix, head and neck and human pluripotent stem cells (which includes both pluripotent stem cells and embryonic stem cells) that model these said diseases.

For lymphangioleiomyomatosis (LAM) used as an exemplary lung disease model, the hydrogel is crosslinked with a peptide that can be degraded by matrix metalloproteinase (MMPs), which is a proteases secreted by LAM cells^{[17]}. In one embodiment, hyaluronic acid (HA) is crosslinked with bismaleimide-terminated collagen I—derived peptide crosslinkers (GPQG-IWGQ) that can be enzymatically degraded by MMPs, enabling MMP-mediated cell invasion into the hydrogels.

Native lung tissue exhibits viscoelastic behaviors, in which structural deformations of the tissue occur to dissipate energy (i.e. stress relaxation) in response to an applied stress force. This occurs via the reorganization of collagen and elastin fibers that comprise the lungs.^{[18]} Thus, we include a second polymer with well-characterized viscoelastic properties (such as methylcellulose) into our 3D hydrogel system that can form weak, reversible physical crosslinks (i.e. via hydrophobic interactions between the methoxy groups of the methylcellulose polysaccharide backbone) to permit cultured cells to remodel the material. To ensure that methylcellulose is retained for the duration of our assay, we chemically modified methylcellulose with reactive thiols (MC-SH, 5% degree of substitution) that form chemical crosslinks with maleimide-functionalized peptides via conjugate Michael addition chemistry (FIG. 1B, 1C). Inclusion of MC-SH into the hydrogel platform increased stress relaxation, but did not significantly affect Young's modulus, as determined by unconfined compression testing (FIG. 1D, 1E).

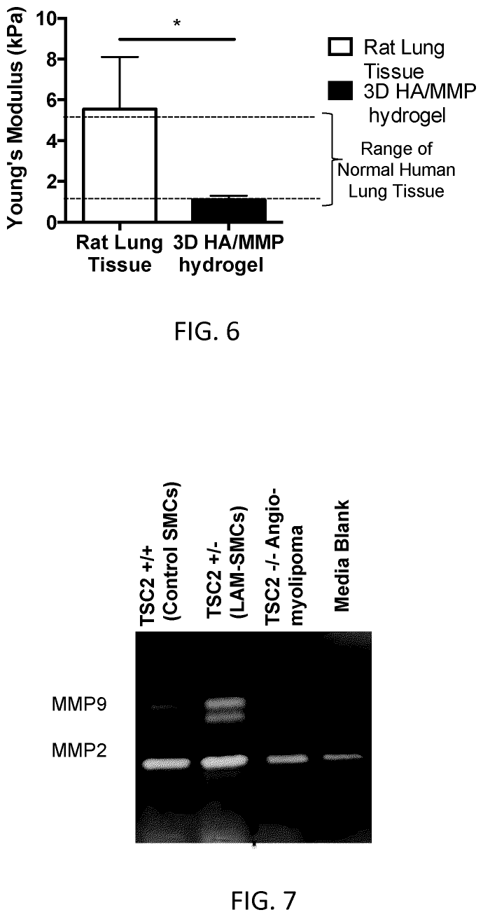

The inventors further compared the mechanical properties of these hydrogels (which we optimized to enable cell invasion) to that of the rat lung (1.10±0.20 kPa for HA-MMP crosslinked hydrogel vs. 5.54±2.55 kPa for rat lung tissue, FIG. 6), both of which are within the range of other reports of native human lung tissue (1-5 kPa),^{[19]} and orders of magnitude lower than conventional 2D TCPS (>1 GPa).

To further enhance cell interaction with the matrix through other cancer-associated integrin receptors expressed on the cell surface (i.e., integrins αvβ3),^{[20]} we immobilized the corresponding ligand (i.e. vitronectin peptide, maleimide-PQVTRGDVFTMP)^{[21]} into the gels via conjugation to the unreacted furanyls in the HA backbone.

The hyaluronan hydrogel-based platform favors cell invasion of TSC2^{+/-} LAM-SMCs (FIG. 1F) over healthy TSC2^{+/+} control SMCs, thereby reflecting what is observed clinically (FIG. 1G). To gain greater insight into how our hydrogels can be used to study cell invasion mechanisms such as MMP-dependent and independent pathways, we characterized several aspects of cellular invasion using pharmacological treatment and varying composition of our 3D hydrogel. Using standard gelatin zymography, we detected increased levels of MMP9 secreted by LAM-SMCs compared to control SMCs and transformed angiomyolipoma (TSC2^{-/-}) cells (which exhibit a subset of LAM-associated phenotypes). Similarly, LAM-SMCs increased MMP2 compared to TSC2^{-/-} angiomyolipoma cells (FIG. 1H, FIG. 7).

The small amount of MMP2 detected in the media-only control is attributed to the presence of 1% fetal bovine serum (FBS) used in the culture media.^{[22]} These data demonstrate that our 3D hydrogels differentiate the MMP-dependent invasive behaviors between patient-derived LAM-SMCs, control SMCs, and the angiomyolipoma TSC2^{-/-} cell line, further validating this model to study LAM. Moreover, treatment of the invasive LAM-SMCs with the pan MMP inhibitor (GM6001, 10 μM) resulted in modest, yet statistically significant decrease in cell invasion (FIG. 1I), suggesting that mechanisms other than MMP secretion alone mediate invasion.

The inventors questioned whether MMP-independent mechanisms are required for LAM-SMC hydrogel invasion given that viscoelastic matrices, which can be remodeled or deformed (by stress-relaxation), increase cell mobility and adhesion compared to elastic matrices.^{[3c, 23]} We show that LAM-SMCs, but not control cells, exhibit increased invasion (p<0.001) in the presence (vs. absence) of the viscoelastic polymer MC-SH (FIG. 1J), which has increased stress relaxation.

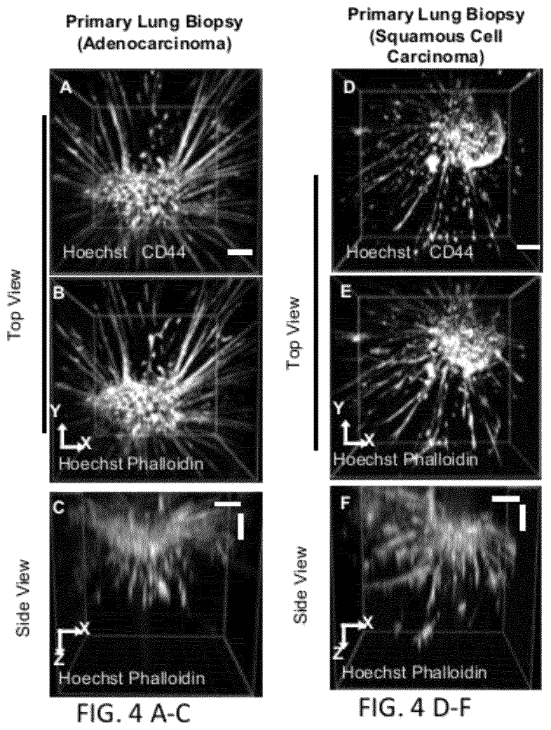

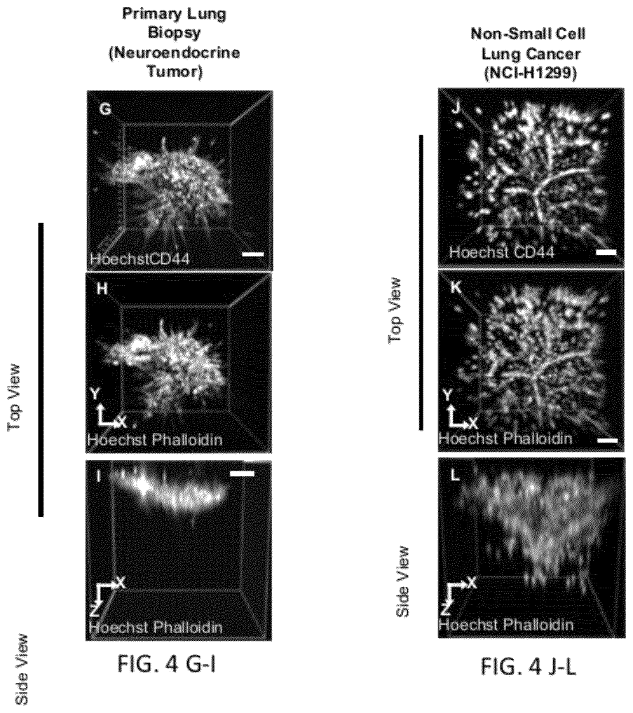

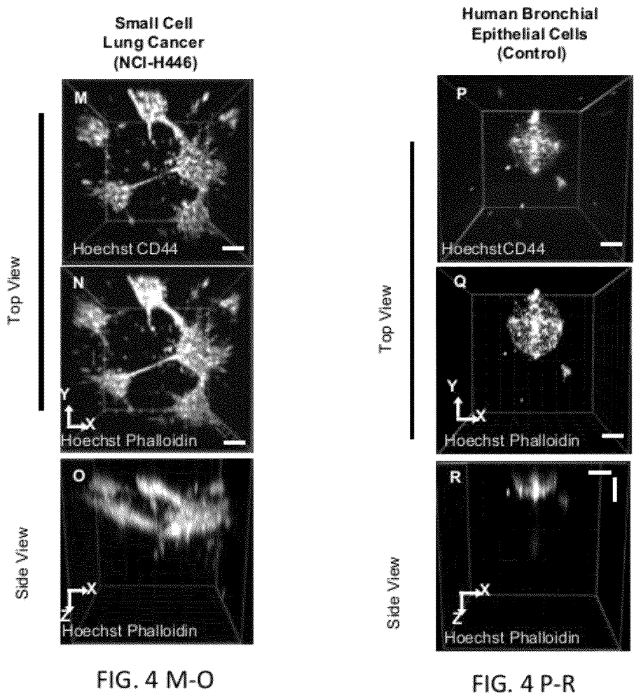

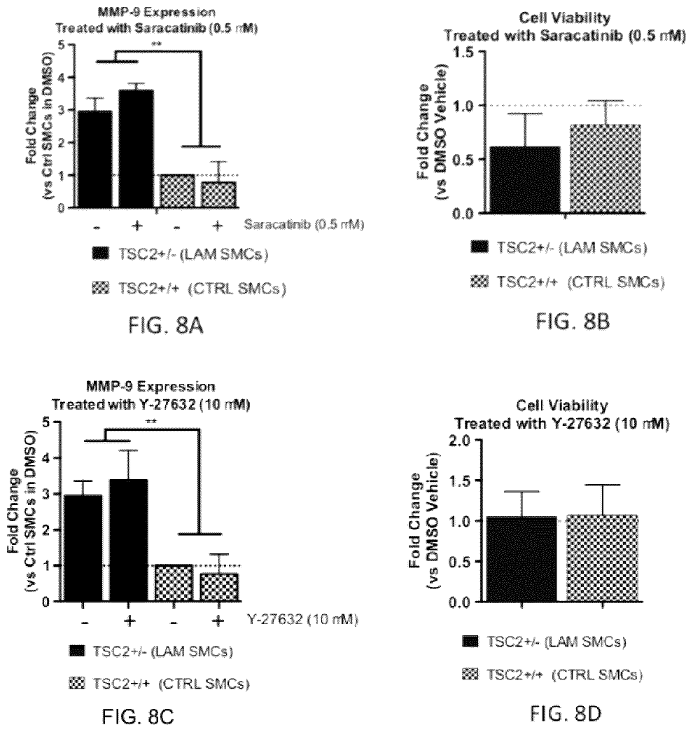

To further assess MMP-independent cell invasion into our 3D hydrogels, cells were treated with the Src inhibitor, Saracatinib, and the ROCK inhibitor, Y-27632 (FIG. 1K). Src kinase, in conjunction with integrin β1 upregulation, is critical for invadopodia formation in MMP-independent cell invasion through the ECM.^{[1b, 24]} Saracatinib (0.5 μM) significantly decreased the relative percentage of invasive LAM-SMCs compared to DMSO-treated controls (FIG. 1K) while not significantly affecting MMP9 secretion or cell viability (p>0.05, FIG. 8A, 8B), demonstrating MMP-independent cell invasion. RhoA activation of cytoskeletal contraction is another MMP-independent mechanism.^{[1b]} Interestingly, LAM- and control-SMCs treated with Y-27632 , an inhibitor of Rho-associated protein kinase (ROCK), significantly decreased cell invasion (p<0.01, FIG. 1K), although neither cell viability nor MMP9 secretion levels of LAM-SMCs were affected (p>0.05, FIG. 4C, 4D). This further substantiates that MMP-independent mechanisms also contribute to LAM cell invasion. Other examples of the inhibitors that can be used according to the embodiments of the present disclosure include:
ABL1
ADENOSINE DEAMINASE
AKT3
ALK
ANDROGEN
AROMATASE
AURORA KINASE
BCL-2
BRAF
BRD
BTK
CALCINEURIN
CCR5
CDK
CXCR
CYTOCHROME P450
DAGK
DNA METHYLTRANSFERASE
DNA TOPOISOMERASE
EGFR
EPH
ERK
Fibroblast Growth Factor Receptors
FARNESYLTRANSFERASE
FLT
FRAP
GSK3
HDAC
HEAT SHOCK PROTEIN
HEDGEHOG
ITGB1
IRE1
JAK2
KDR
KINESIN-LIKE SPINDLE PROTEIN
KIT
LCK
LIMK1
LYN
MAP2K
MDM2
P38B
P70S6K
PARP
PDGFR
PI3K
PKC
PLK1
PIM2
PROTEASOME
RAF1
RET
SIRT2
SPHINGOSINE KINASE
TANKYRASE
TUBULIN
WNT.
   In a further embodiment of the present disclosure, the cell can be treated with any one or a combination of the following agonists:
GLUCOCORTICOID
PKM2
PROGESTERONE
RXR
S1P RECEPTOR.

Together, HA and MC polysaccharides form a stable hydrogel for cell-mediated invasion that is both MMP-dependent—by degradation of MMP-cleavable crosslinks between HA chains, and MMP-independent—by physical displacement of reversible hydrophobic interactions between MC chains.

Immunocytochemistry of LAM lesions^{[25]} suggests that in addition to TSC2^{-/-} cells, TSC2^{+/-} SMCs play a pathophysiological role in LAM lesions. We determined how TSC2 gene expression is affected by the substrate on which the cells are cultured and hypothesized that TSC2^{+/-} LAM-SMCs cultured in our biomimetic hydrogel would behave more similarly to in vivo LAM cells. We previously reported that TSC2^{+/-} LAM-SMCs express decreased levels of TSC2 at both mRNA and protein levels compared to TSC2^{+/+} control SMCs when cultured on 2D TCPS.^{[12a]}

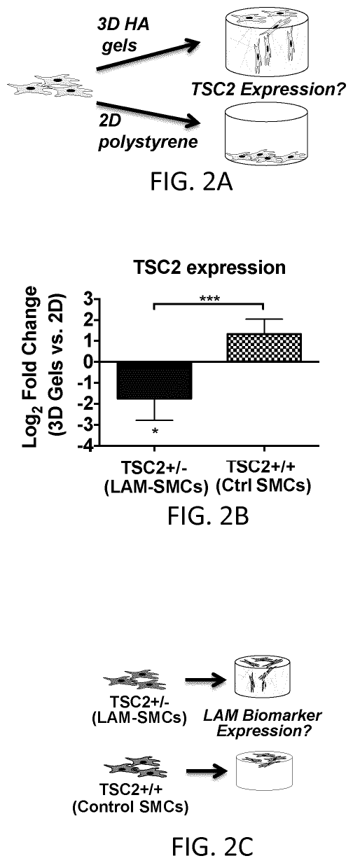

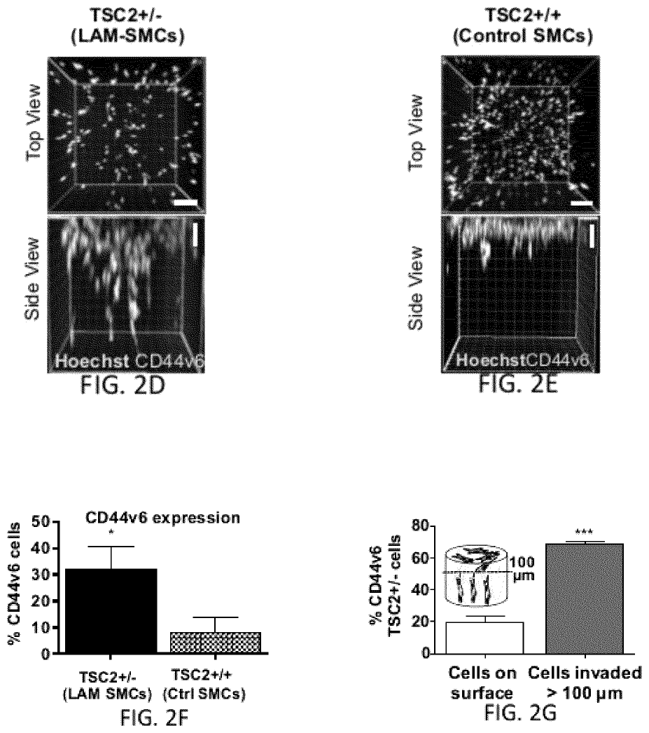

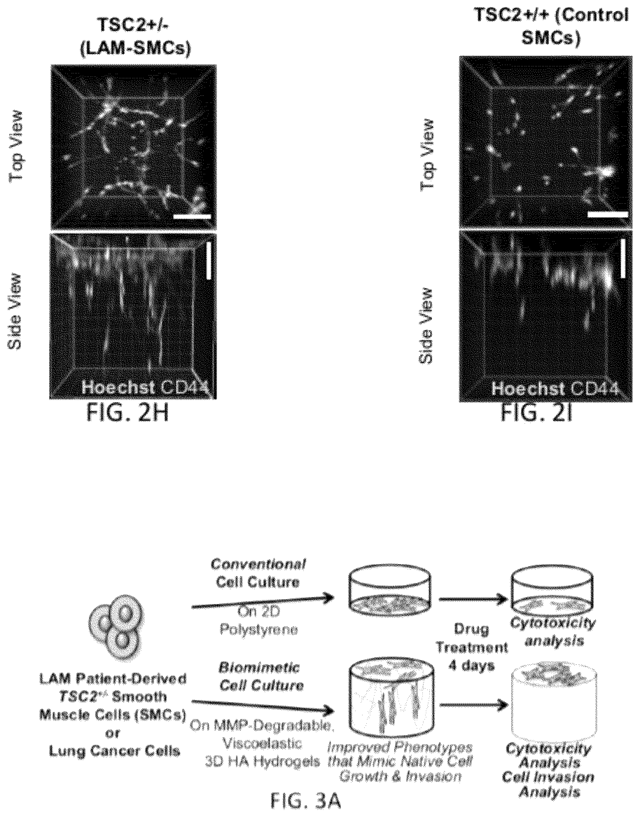

To assess the impact of cell-substrate interactions, we quantified TSC2 levels by performing qRT-PCR of patient-derived TSC2^{+/-} LAM-SMCs and TSC2^{+/+} control SMCs cultured on either stiff 2D TCPS or soft biomimetic 3D HA hydrogels (FIG. 2A, 2B). LAM-SMCs cultured on 3D HA hydrogels express decreased TSC2 transcript compared to those cultured on 2D TCPS (p<0.01). Surprisingly, TSC2^{+/+}control SMCs increased TSC2 mRNA levels when cultured on 3D hydrogels compared to 2D (p<0.05), revealing an even greater difference between control and patient-derived LAM-SMCs in our biomimetic 3D HA hydrogels than on 2D TCPS (p<0.001), thereby highlighting the importance of growing cells in tissue-mimetic 3D conditions to model disease.

To gain insight into the interactions between the 3D HA matrix and cells cultured therein, we assessed the cell surface abundance of CD44 and CD44v6 receptors that naturally bind to HA and are upregulated in many cancer cells,^{[26]} including primary cells isolated from the lungs of LAM patients.^{[14]} TSC2^{+/-} LAM-SMCs showed markedly increased expression of a variant of CD44 that is associated with LAM and cancer cell invasion (CD44v6) compared to control SMCs (FIG. 2D-2F, p<0.05), consistent with immunohistochemistry of primary LAM nodules. We quantified the degree of CD44v6⁺ cells based on their invasiveness into the hydrogels (FIG. 2G): cells that invade greater than a depth of 100 μm show increased CD44v6 expression compared to non-invasive cells (FIG. 2D, 2G, p<0.001), further demonstrating the importance of this cell-surface marker as an indicator of invasiveness of LAM cells. Unlike CD44v6 , CD44 is expressed in iPSC-derived SMCs of both LAM patients and normal controls regardless of their invasiveness (FIG. 2H, 2I).

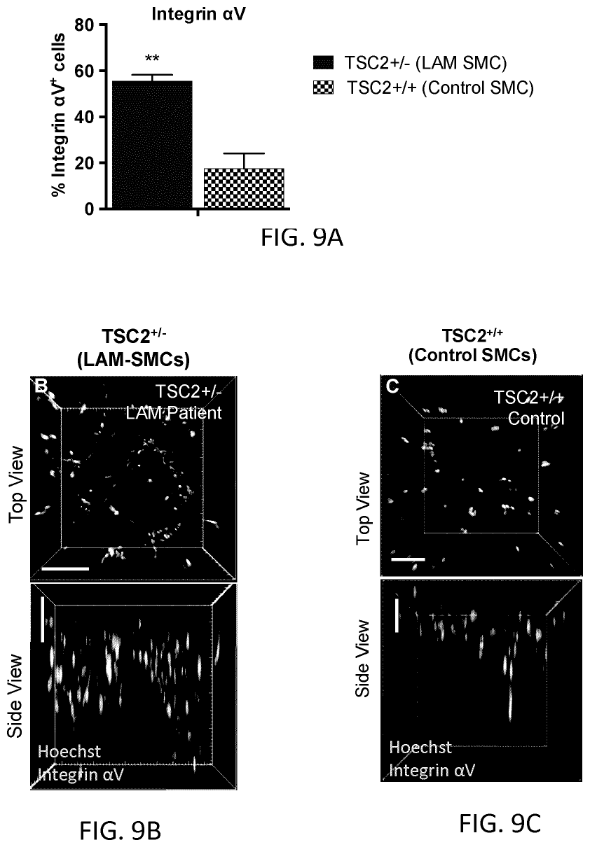

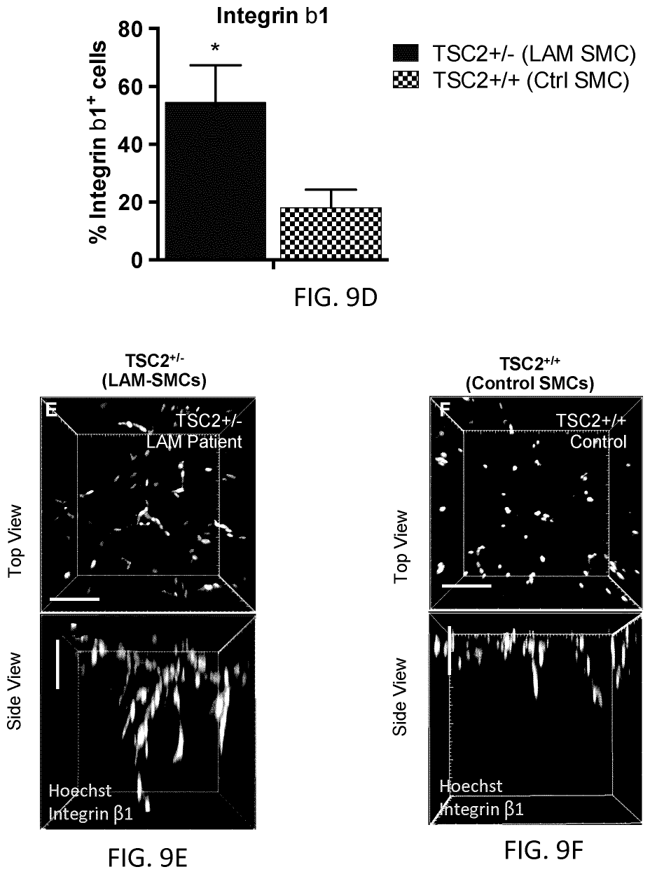

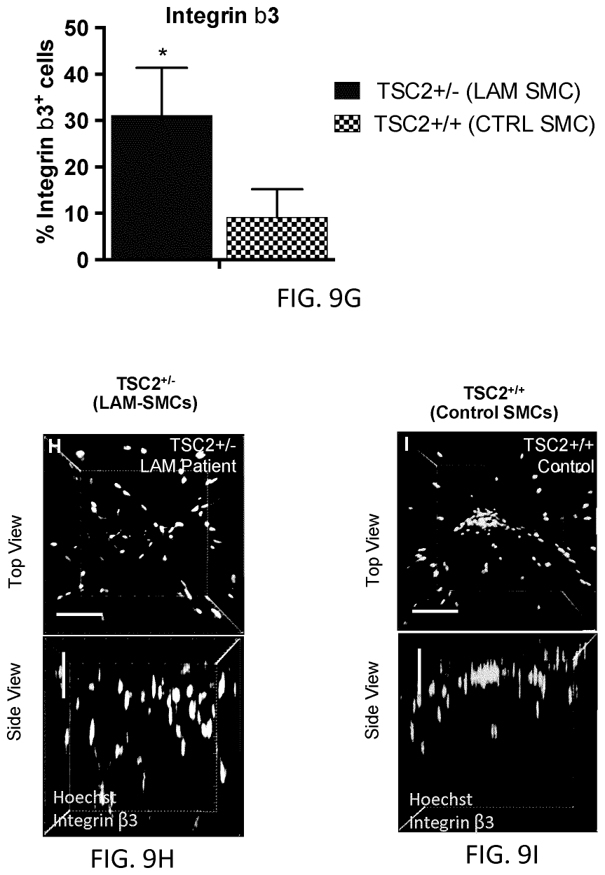

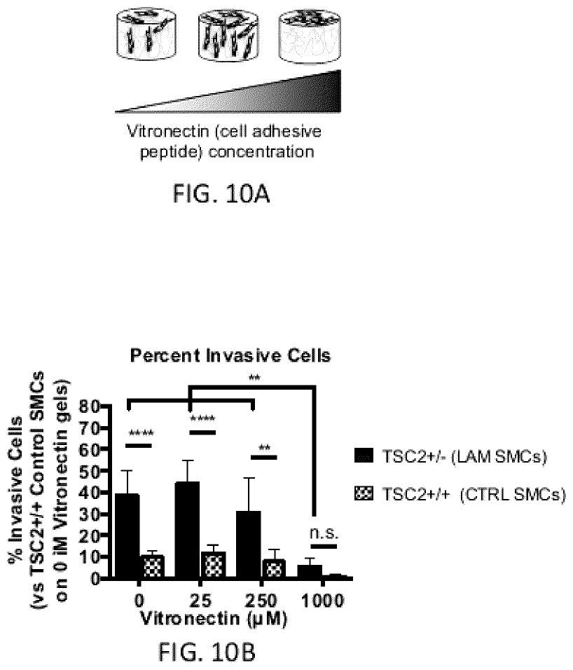

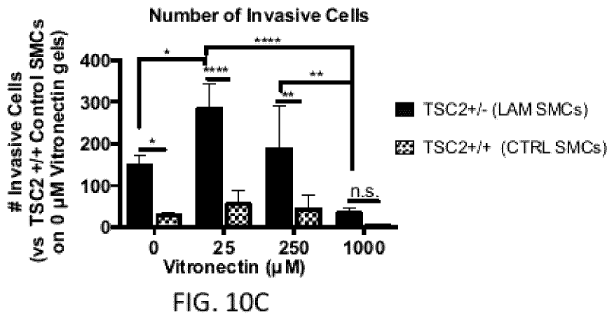

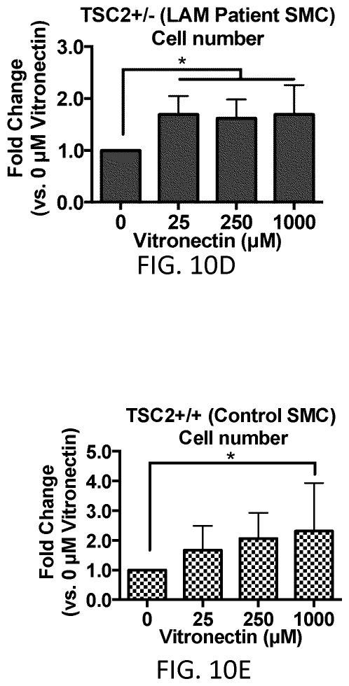

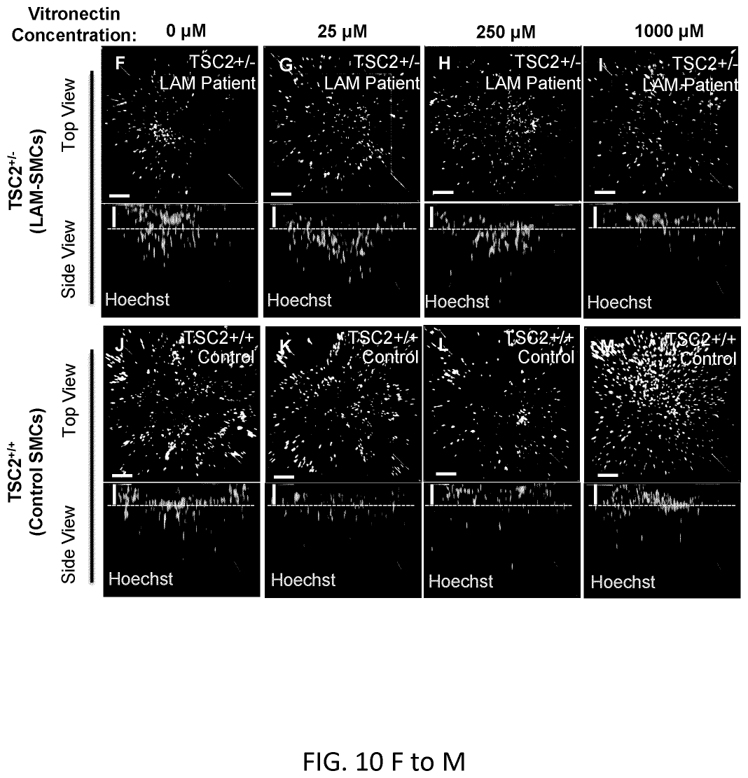

We demonstrate the role of vitronectin in our hydrogel platform by first confirming that LAM-SMCs express higher levels of the vitronectin-interacting integrin subunits αV, β1 and β3 compared to control SMCs (FIG. 9A-9I). By varying vitronectin concentrations, we observe that an optimal vitronectin peptide concentration of 25 μM promotes the greatest differences in cell invasion between LAM and control SMCs (FIG. 10). While the overall percentage of invasive cells is comparable between hydrogels immobilized with 0, 25 or 250 μM of vitronectin peptide (p>0.05, FIG. 10B), we observe the greatest statistical differences in the number of invasive cells between the two cell types at 25 μM compared to 0 and 250 μM (p<0.0001, p<0.05, p<0.01 for 25, 0, 250 μM, respectively, FIG. 10C). The absence of vitronectin peptide results in an overall decrease in cell number for LAM-SMCs, and consequently a decrease in the number of invasive cells (p<0.05, FIG. 10C). Conversely, both LAM and control SMCs cultured on gels with the highest peptide concentration (1000 μM) formed monolayers on top of the gels (FIG. 10C, 10I, 10M) with minimal invasion.

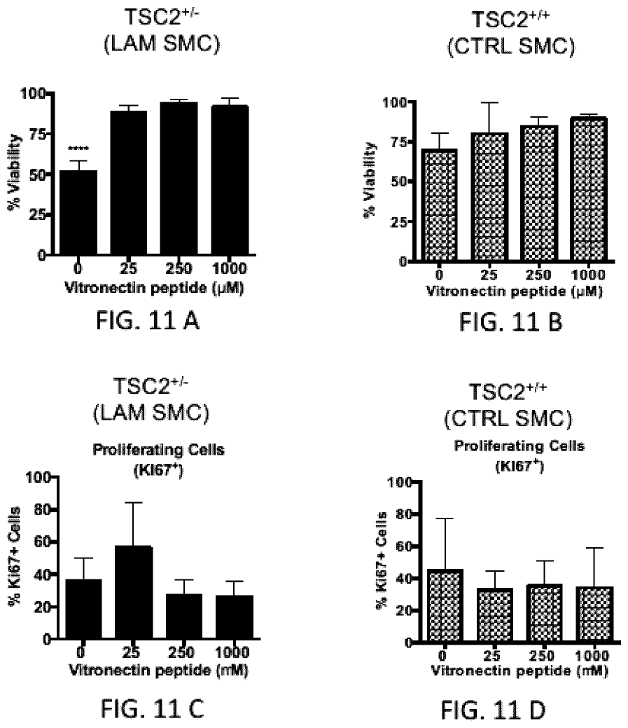

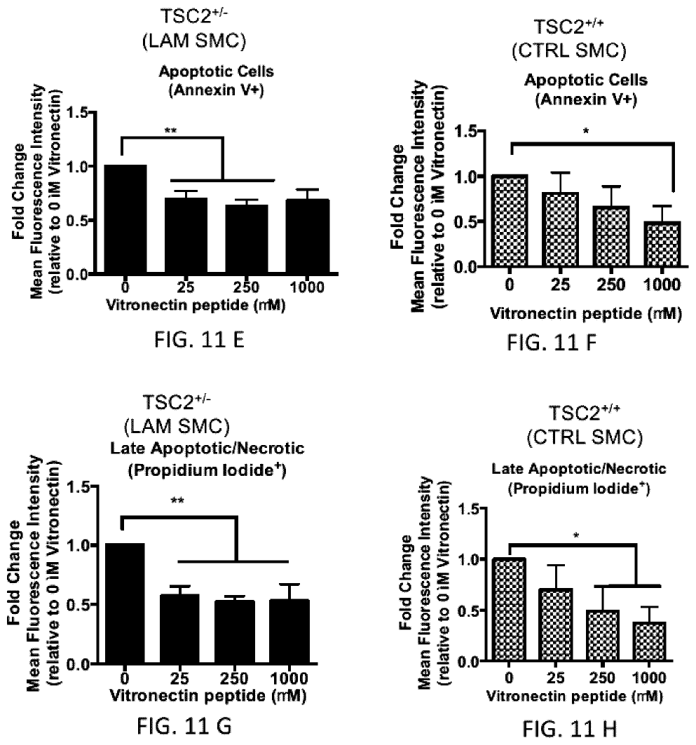

To gain further insight into the differences between the percentage and number of invasive cells (FIG. 10B, 10C) at 0 and 25 μM, we studied the viability (FIG. 11) of cells cultured in hydrogels immobilized with 0, 25, 250, and 1000 μM of vitronectin peptide. We observed that while cell proliferation (Ki 67⁺ cells) is not statistically different between cells grown in 0 to 1000 μM vitronectin, the lack of peptide resulted in decreased cell viability (Calcein AM⁻ staining) and increased early and late apoptosis (Annexin V⁺ and propidium iodide (Pi)⁺ cells, respectively) of LAM SMCs. Therefore, with our goal in using a hydrogel system as a platform that can delineate differences in cell invasion between LAM and control SMCs for drug screening applications, we performed subsequent drug screening experiments for both cell viability and invasion using 25 μM.

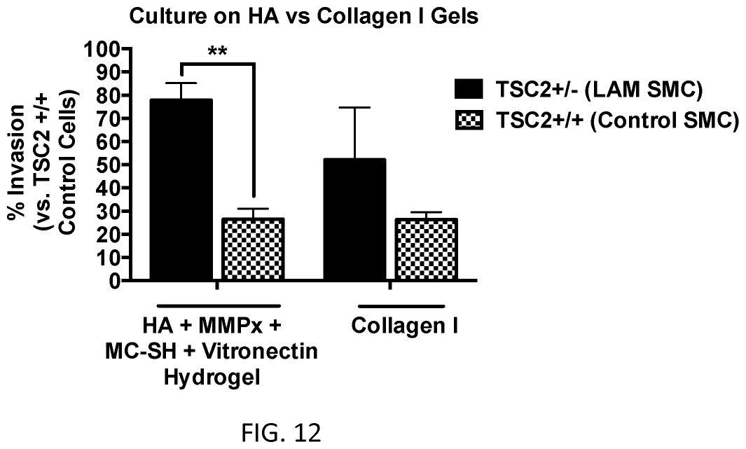

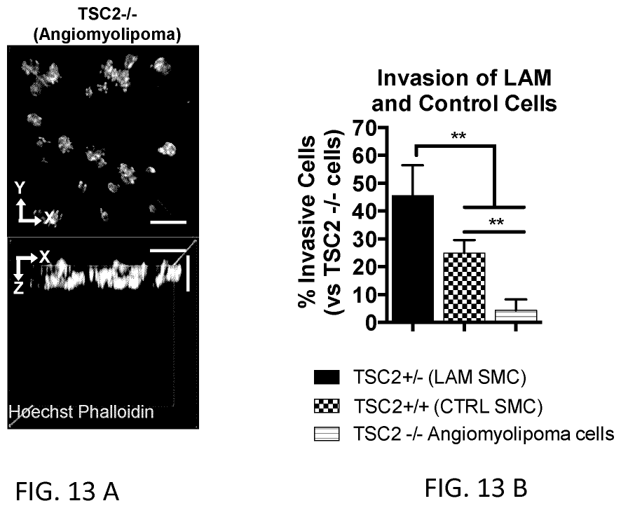

To test whether our hydrogel platform is optimized for performing high-content drug screening of cell invasion and viability, we compared our strategy to previously reported methods used to study LAM cell invasion, such as cell culture in conventional collagen I hydrogels and the use of angiomyolipoma (TSC2^{-/-}) cells.^{[11a, 27]} In comparison to collagen I, we observed a greater difference in our HA hydrogels between TSC2^{+/-} LAM-SMCs and TSC2^{+/+} control SMCs (FIG. 12). Surprisingly, both cell types showed significantly greater invasion compared to TSC2^{-/-} angiomyolipoma cells (p<0.01, FIG. 13), reflecting the superiority of both our hydrogel and cells to model LAM.

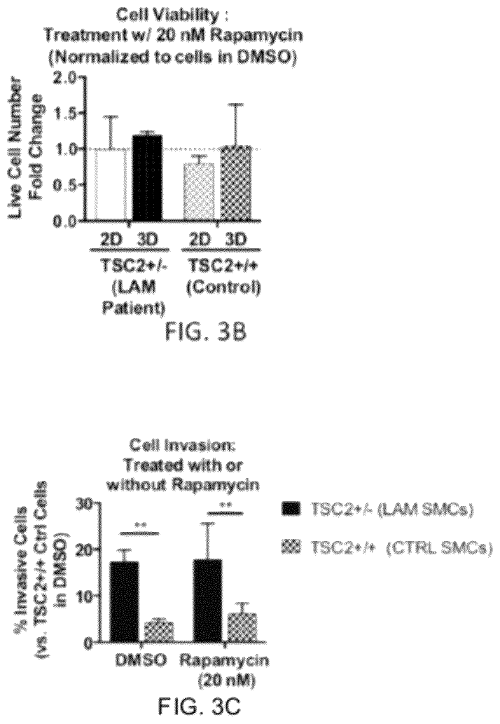

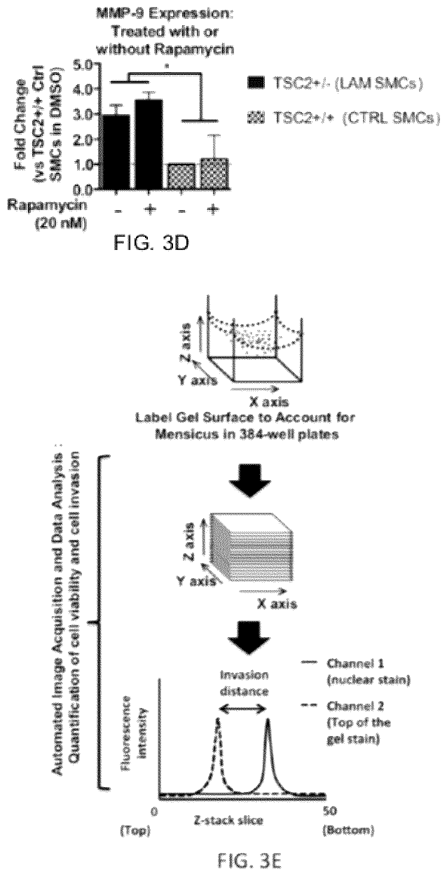

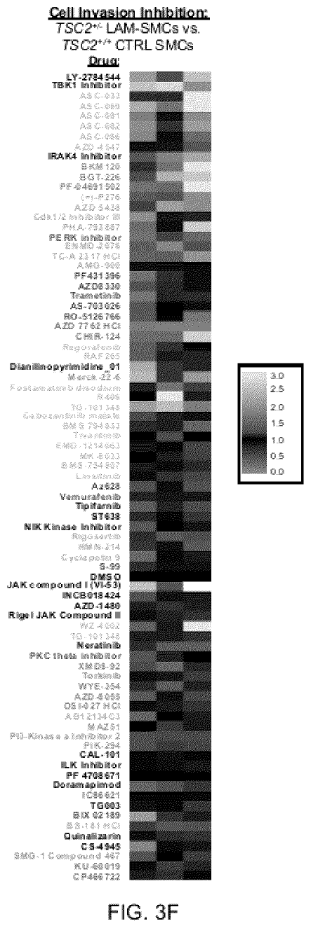

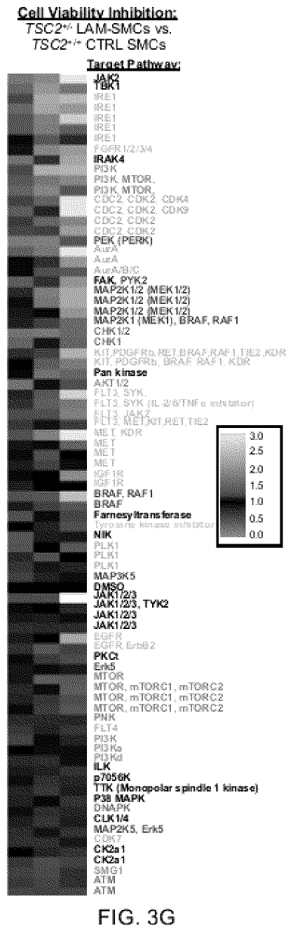

For invasive diseases, both cell viability and invasion are key outcome measures of drug therapies. Currently, the only approved therapeutic treatment for LAM is the mTORC1 inhibitor rapamycin,^{[28]} which slows the decline in lung performance of LAM patients, but as a cytostatic and not cytotoxic agent, rapamycin has limited effectiveness. Upon withdrawal of rapamycin, lung function decreases comparably to placebo-treated control patients, emphasizing the need to discover more efficacious drugs. We tested the efficacy of rapamycin in our 3D hydrogel platform: treatment with rapamycin at 20 nM (and up to 1 μM) had little effect on the viability of either patient-derived TSC2^{+/-} LAM-SMCs or TSC2^{+/+} control SMCs (FIG. 3B) cultured on either 2D TCPS or 3D gels, thereby corroborating the patient data.^{[27-28]} Moreover, rapamycin treatment neither diminished cell invasion of LAM-SMCs (FIG. 3C, p=0.998) nor decreased MMP expression (FIG. 3D, p=0.543).

Next, we incorporated our hydrogel into a drug-screening platform and tested drug response between TSC2^{+/-} LAM-SMCs and healthy TSC2^{+/+} control SMCs. We modified our culture system to a 384-well format to enable higher throughput screening and simultaneous quantification of cell invasion and cell viability. At the endpoint of the assay (4 days), cell viability is determined by staining with both Hoechst (for cell nuclei) and SyTox Green (for dead cells) while cell invasion is measured by automated confocal imaging: the hydrogel surface is demarcated using silica gel particles to accurately account for the gel-surface meniscus present in 384-well plates and z-stacked images are obtained for each well using an automated confocal high content imaging system (FIG. 3E). This system will work in other multiwell (or single well) plates, including plates with both smaller (i.e., 1536) and larger (i.e., 96, 48, 24, 6) wells.

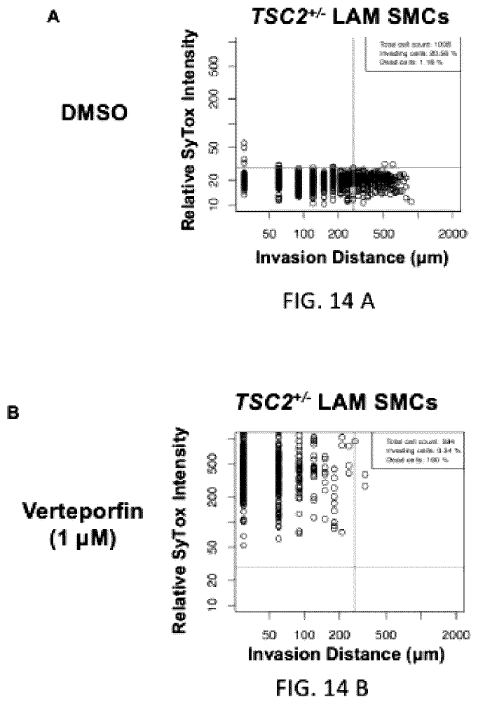

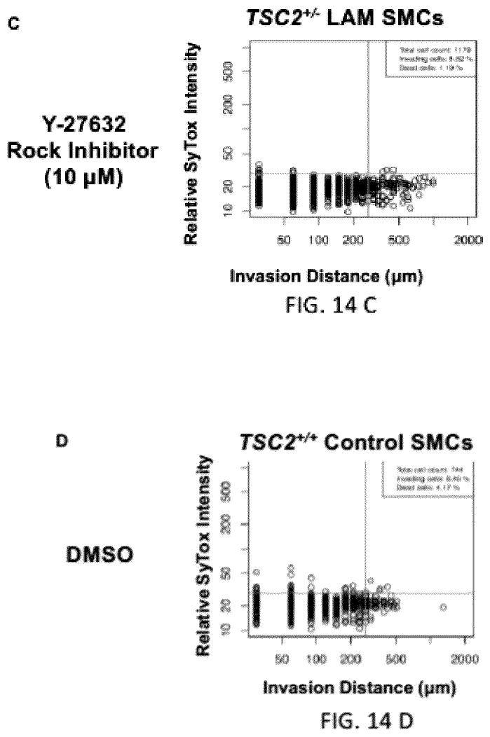

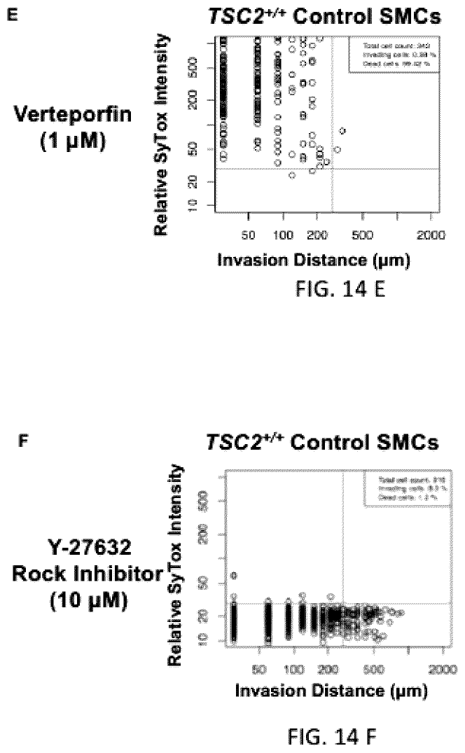

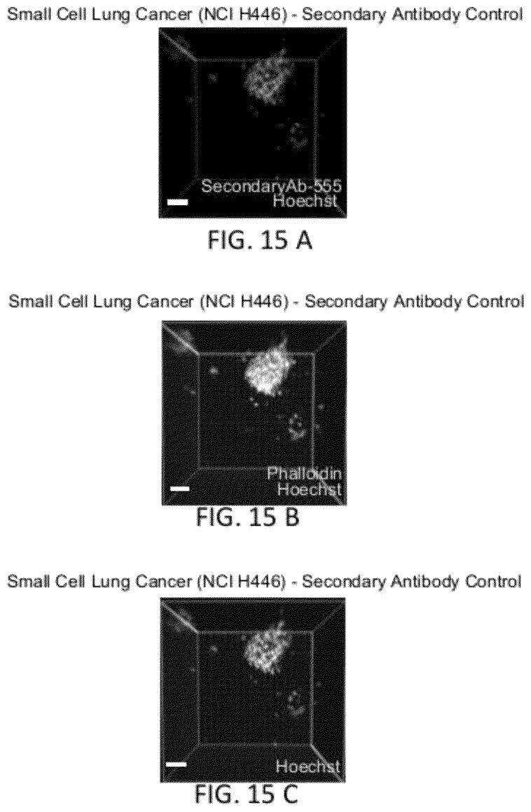

The inventors have developed a novel algorithm in ImageJ to quantify cell invasion from the surface of the hydrogels by subtracting the Z-position of the cell nuclei from the Z-position of silica gel particles (i.e. at the gel surface) at the same XY coordinates. Together with identification of dead cells by staining with SyTox Green, this method enables independent quantification of viability and invasion of individual cells (FIG. 14).

We used our 3D hydrogel platform to simultaneously and independently assess cell invasion and viability of a panel of 80 kinase inhibitors (FIG. 3F, 3G), thereby identifying potential drug candidates and target pathways towards TSC2^{+/-} LAM-SMCs vs. TSC2^{+/+} control SMCs. Treatment with drugs that showed selective decrease in both cell viability and invasion towards LAM-SMCs (FIG. 3F, 3G) include those that affect: (1) cell cycle—i.e., cyclin-dependent kinase inhibitors: Cdk1/2 inhibitor, PHA-793887, AZD5438 and (+)-P276; Aurora A inhibitors: ENMD-2076, TC-A 2317 NCI; and (2) autophagy—i.e., IRE1 inhibitors: ASC-033, ASC-069, ASC-081, ASC-082 and ASC-086, indicating that these specific pathways represent pharmacological targets in pulmonary LAM. Surprisingly, drugs that directly targeted the mTOR pathway (which is downstream of TSC2) did not consistently inhibit both invasion and viability of hypomorphic TSC2^{+/-} LAM-SMCs, suggesting the importance of targeting mTORC1-independent pathways in LAM.

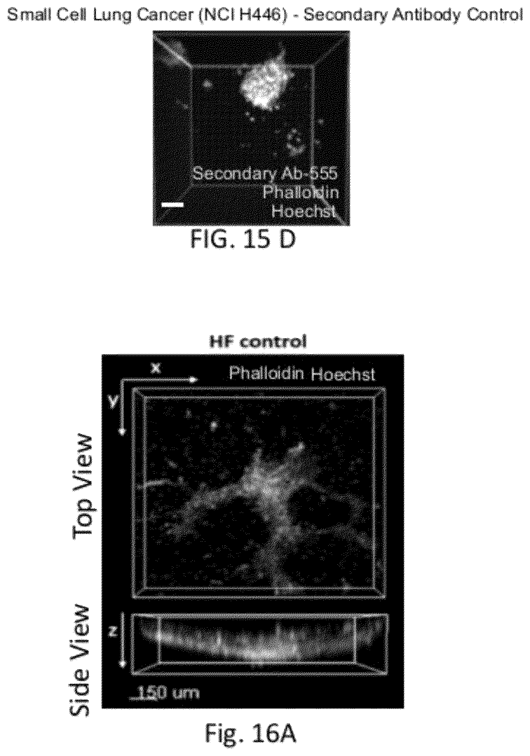

To test the broad utility of our hydrogel platform, multiple lung cancer cells were cultured on our HA hydrogels (FIG. 4A-4O). Three distinct patient-derived primary lung cancer cell preparations, isolated from three separate lung cancer tissue biopsies and identified as adenocarcinoma (FIG. 4A-4C), squamous cell carcinoma (FIG. 4D-4F), and neuroendocrine tumor (FIG. 4G-4I), along with commercially available human non-small cell lung cancer (NSCLC, NCI-H1299, FIG. 4J-4L) and small cell lung cancer (SCLC, NCI-H446) cells (FIG. 4M-4O) all express CD44 (FIG. 4A, 4D, 4G, 4J, 4M, FIG. 15), which is the natural ligand for HA. Interestingly, CD44 is predominantly expressed on cells that are at the cell-matrix interface (i.e. on the outside of multicellular cell clusters and on single cells), yet is not readily detected in cells within the cell clusters, consistent with this receptor interacting with the HA hydrogel. However, healthy human bronchial epithelial control cells do not express CD44 (FIG. 4P), further highlighting the advantage of using HA-based hydrogels to culture lung cancer cells.

Confocal imaging analysis revealed different cell morphologies and levels of invasiveness, which cannot be assessed with conventional 2D cell culture or Boyden chamber/transwell assays. Cells isolated and cultured from three lung biopsies formed large cell clusters with spindle-like cells migrating away from these clusters; however, only cells from adenocarcinoma and squamous cell carcinoma biopsies showed high degrees of cell invasion (FIG. 4C, 4F), whereas cells grown from a neuroendocrine biopsy showed less invasion (FIG. 4I). Interestingly, non-small cell lung cancer (NCI-H1299) cells grew and invaded as single cells (FIG. 4K, 4L), whereas SCLC cells formed interconnected multicellular spheroids from which single cells also invade into the hydrogels (FIG. 4N, 4O). In contrast, healthy bronchial epithelial control cells do not invade and instead remain on the surface of these hydrogels as spherical aggregates (FIG. 4Q, 4R).

The present disclosure also demonstrates its utility in a brain disease model, using glioblastomas (GBM) as an exemplary embodiment. As discussed in more detail under the “Example”, hydrogels were prepared by functionalizing relevant ECM molecules in the GBM microenvironment, namely hyaluronan, methylcellulose, adhesive peptides and enzymatically degradable peptides, with mutually reactive Diels-Alder functional groups. The biomimetic platform thus produced was then used to culture Patient-derived GBM cell lines and healthy human fetal neural stem cells (HFNSC's, a negative control).

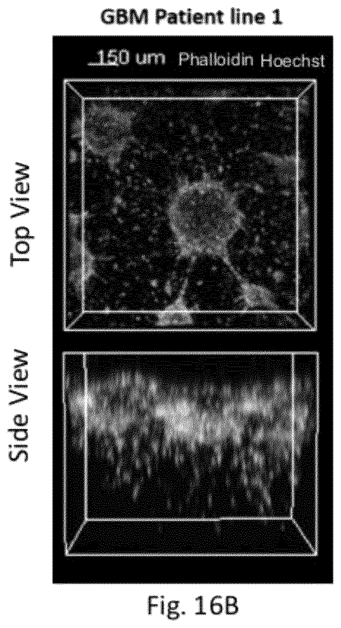

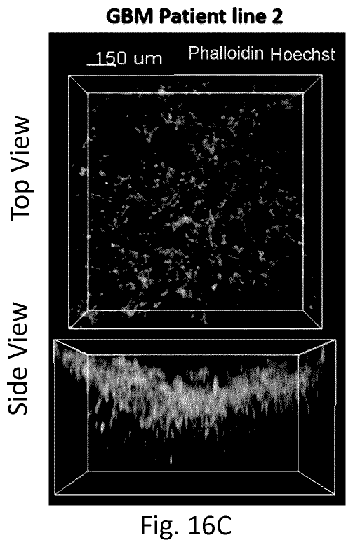

Referring to FIG. 16, two patient derived cell lines tested invaded into the hydrogels, while the human fetal line grew as a non-invading monolayer on the hydrogel. Quantitative analysis of these images demonstrated a significant increase in percentage of invading cells and invasion depth for the patient derived lines compared to the HFNSC control.

Integrin beta 1 (ITGB1) is involved in cellular binding to many extracellular matrix components, including fibronectin,[29] and ITGB1 gene knockdown has been shown to reduce invasion in cancer cells.[30] Thus, we hypothesized that ITGB1 knockdown would reduce the invasive behavior of glioblastoma stem cells (GSCs) isolated from brain cancer patients.

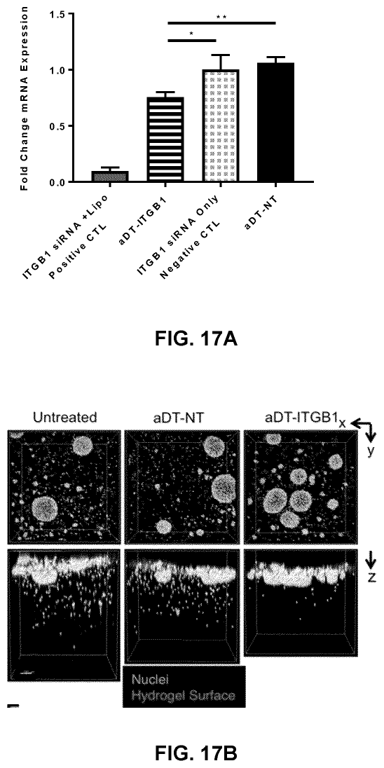

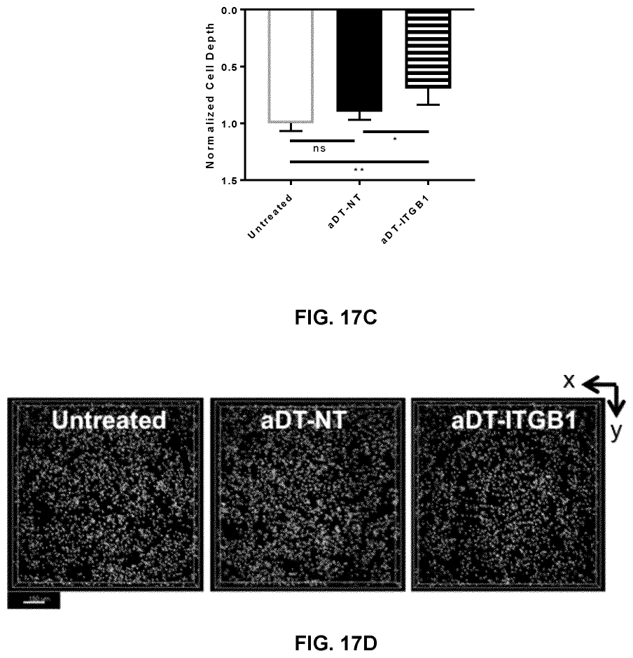

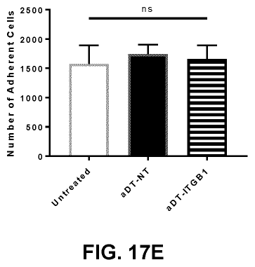

To knockdown the ITGB1 gene, we conjugated a Dicer-substrate siRNA against the gene target ITGB1 to an attenuated diphtheria toxin (aDT) delivery vehicle (to make aDT-ITGB1). We treated the GSCs with the aDT-ITGB1 conjugate and observed a significant reduction in the target mRNA compared to negative controls of siRNA only (without lipofectamine) and aDT conjugated to a non-targeting siRNA (aDT-NT) at 50 nM (FIG. 17A). To ascertain whether the reduction in ITGB1 expression would correspond to a phenotype of either reduced invasion or adhesion, we seeded the cells on top of the 3D hydrogel described in this disclosure (FIG. 17B). Impressively, we observed a striking decrease in invasiveness in aDT-ITGB1-treated cells compared to untreated or aDT-NT controls (FIG. 17C, D). To determine whether cell adhesion influenced these results, we pre-treated the cells cultured in 2D tissue culture polystyrene flasks with both aDT-ITGB1 and aDT-NT prior to plating them on the 3D hydrogels. After several wash steps, we observed no significant difference in the number of adhered cells between any of the treatment groups, demonstrating that cell adhesion did not impact the reduced cell invasion observed with aDT-ITGB1 treatment (FIG. 17E, F). Together, these data demonstrate that the gels described in this disclosure can be used to test functional effects of siRNA-mediated knockdown (such as the effectiveness of reducing cell invasion using aDT-ITGB1 siRNA delivery vehicles).

Non-limiting examples of furanyl groups include 2-methyl furan.

### EXAMPLES

The following give detailed description of some non-limiting exemplary embodiments of the present disclosure, including material, but are not meant to be limiting.

### Materials

Sodium hyaluronate (HA, 242 kDa) powder, was purchased from

Lifecore Biomedical (Chaska, Minn., USA). 4-(4,6-Dimeth-oxy-1,3,5-triazin-2-yl)-4-methyl-morpholinium chloride (DMTMM), dimethyl sulfoxide (DMSO), diisopropylcarbodiimide (DIC), borane dimethylamine complex, and triisopropylsilane were purchased from Sigma-Aldrich (St. Louis, Mo., USA). Tetrakis(triphenylphosphine)palladium was purchased from TCI America (Philadelphia, Pa., USA). Maleimide propanoic acid was purchased from Toronto Research Chemicals (Toronto, Canada). Amino acids and reagents for peptide synthesis were purchased from Anaspec (Fremont, Calif., USA). Furfurylamine was purchased from Acros Organics (New Jersey, N.J., USA). 2-(N-Morpholino)-ethanesulfonic acid (MES) was purchased from Bioshop Canada Inc. (Burlington, ON, Canada). Dulbecco's phosphate buffered saline (dPBS) was purchased from Multicell Technologies Inc. (Woonsocket, R.I., USA). Fluorescently labeled polystyrene microspheres (0.1 μM, orange) was purchased from Phosphorex (Hopkinton, Mass., USA). SiliFlash silica gel particles (40-63 μm, 230-400 mesh) were purchased from SiliCycle.

Phalloidin-AlexaFluor488, goat anti-mouse and anti-rabbit-AlexaFluor 488, and Hoescht 33342 were purchased from Life Technologies (Burlington, Canada). Dialysis membranes were purchased from Spectrum Laboratories Inc. (Rancho Dominguez, Calif., USA). M231 media, smooth muscle growth supplement (SMGS) and gentamycin were purchased from Thermo Fisher Scientific (Waltham, Mass. USA). Human bronchial epithelial cells were generously donated by Christine Bear (University of Toronto). NCI-H1299 (non-small cell lung cancer) and NCI-H446 (small cell lung cancer) cells, and RPMI-1640 media were purchased from ATCC. RPMI-1640 was purchased from CedarLane.

### Peptide Synthesis

Vitronectin-mimetic peptide (maleimide)-KGGPQVTRGDVFTMPKG (1796 gmol⁻¹), fibronectin (CS1 region)-mimetic peptide (maleimide)-DELPQLVTLPHPNLHGPEILDVPSTG (2940.6 gmol⁻¹), MMP-degradable peptide crosslinker (maleimide)-KKGRGPQGIWGQKGPQGIWGQ- K(maleimide)S (2681 g mol⁻¹) were synthesized using microwave-assisted Fmoc solid phase peptide synthesis with a CEM Liberty Blue automated peptide synthesizer.

For MMP-degradable peptide crosslinker, Fmoc-Lys(Alloc)-OH was first immobilized to Fmoc-Gly-Wang resin using manual solid phase peptide synthesis. Briefly, to 1.0 mmol of deprotected Gly-Wang resin, 3.0 mmol Fmoc-Lys(Alloc)-OH was pre-activated with HBTU (3.5 mmol) in a 3:1 mixture of dichloromethane (DCM):N-methyl pyrrolidinone (NMP) for 15 min. This mixture was then added to the pre-swollen Wang resin and 6.0 mmol diisopropylethylamine (DIPEA) was added and mixed overnight. Completion of the coupling reaction was monitored using the 2,4,6-trinitrobenzene sulfonic acid (TNBS) test. The remainder of the amino acids were coupled to the resin using standard Fmoc chemistry on a CEM solid phase peptide synthesizer. Following coupling of the final amino acid, the amine was kept protected with an Fmoc group. To functionalize the C-terminus of MMP-degradable peptides, further modifications were performed manually. The C-terminal allyloxycarbonyl (alloc) groups were cleaved using Pd(PPh₃)₄ (cat.), borane dimethylamine complex (10 eq.) in DCM, under N_{2G} overnight at room temperature. The resin was washed extensively with methanol and then DCM.

For both MMP-degradable, vitronectin- and fibronectin-mimetic peptides, the N-terminal Fmoc was deprotected using 20% piperidine in DMF. Maleimide propanoic acid (2.5 eq. relative to free amines) was diluted in a 3:1 mixture of DCM:NMP, and activated using diisopropylcarbodiimide (10.0 eq. relative to free amines) for 20 min. Diisopropyl urea byproducts were removed by filtration, and the resulting filtrate was then added to the peptide resin and mixed overnight. Reaction completion was monitored by performing a TNBS test. Finally, the peptide was cleaved from the resin using a cleavage cocktail comprising 9:1:0.5 (v/v/v) of TFA: triisopropylsilane: H₂O and 10 eq. L-lysine. The resulting crude mixture was precipitated in cold diethyl ether. The resulting precipitate was centrifuged at 2200 RPM for 5 min. The ether washes were decanted and the precipitate was washed two more times with cold diethyl ether and allowed to dry overnight. The peptide was purified using C₈ reversed phase HPLC (mobile phase: 15:85 to 50:50 (v:v) 0.1% TFA in acetonitrile:water gradient over 30 min). Peptides were characterized using mass spectrometry (electrospray ionization).

### Hyaluronan Hydrogel Preparation

HA (230 kDa) was functionalized with furfurylamine as previously described^{[16]} Furanyl-modified HA/MMPx-(maleimide)₂ hydrogels were prepared as previously described ^{[16]} with the following modifications: To prepare 1.0 mL of 0.9% HA hydrogel with 25 μM vitronectin peptide for SMC culture, HA-furanyl (65% furanyl substitution, 9.0 mg HA, 13.3 μmol furanyl) was dissolved in 450 μL of dPBS (adjusted to pH 6.5), and added to 357.7 μL of dPBS (pH 6.5). 150.1 μL of maleimide₂-MMP-degradable crosslinker peptide dissolved in 0.1 M MES buffer (pH 5.5, 50 mg/mL, 2.8 μmol of peptide, 5.6 μmol of maleimide) and 3.75 μL of maleimide-vitronectin peptide dissolved in 0.1 M MES buffer (pH 5.5, 12 mg/mL, 25 nmol) were added to the HA-furanyl solution and carefully mixed to prevent the formation of air bubbles. 38.5 μL of a thiolated methyl cellulose solution (2.6 mg/mL MC-SH in DI H₂O, 100 nmol_{free thiols}/mg_{MC}) was then added to this solution and mixed carefully with a pipette. Hydrogel formulations were tuned for lung cancer cells. For culturing primary cells from lung cancer tissue biopsies, 100 μM vitronectin peptides were used. For NCI-H1299, NCI-H446 and human bronchial epithelial cells, 100 μM vitronectin peptides and 100 μM fibronectin peptides were used. To prepare gels in 384-well plates, 15 μL of the hydrogel solutions were added to each well in a 384-well plate and placed in the incubator overnight at 37° C.

Excess sterile water was then added to wells lining the outside of the plates to prevent hydrogel evaporation. The next day, the gels were washed with PBS (75 μL each time, 2 times, 45 mins each), followed by equilibration in DMEM that does not contain FBS (75 μL, >45 mins). Gels were then equilibrated with either RPMI-1640 containing 1% FBS (for lung cancer cells) or M231 SMC media (75 μL, >45 mins). Media was then removed, and for SMC culture, 15 μL of M231 supplemented with 25% SMGS (containing a final FBS concentration of 1%) was added into each well for at least 1 h prior to cell seeding. For drug treatment assays, this 15 μL volume of M231 with 25% SMGS also includes a 5× concentration of the desired drug. For larger drug screening assays, Y-27632 (Rock inhibitor, 10 μM) was used as a negative control for cell invasion, and verteporfin (1 μM) was used as a positive control for cell toxicity (SyTox Green staining). For lung cancer cell culture, cells in RPMI media containing 1% FBS was added to each well.

### Uncompressed Mechanical Testing

The Young's moduli were determined for HA hydrogels and healthy rat lungs. For HA hydrogels, 75 μL of gels comprising 1.1% HA and 4.18 mM of maleimide₂-MMP-degradable crosslinker peptide were prepared with a 5 mm diameter. Gels were washed and pre-swollen in PBS prior to analysis. For rat lung tissue, lungs were soaked in PBS and then ethanol overnight prior to analysis. A 5 mm biopsy punch was used to isolate sections for mechanical testing. Samples were placed between two impermeable flat platens connected to a 150 g single axis load cell (ATI Industrial Automation) on a Mach-1 micro-mechanical system (Biomomentum), and an initial force of 0.01 N was applied to determine the surface of the sample. The initial sample height was determined as the platen-to-platen separation. An initial uniaxial, unconfined compression was applied at a strain of 10% of the gel height to even out surface defects. Sample analysis was performed by applying 2% strain for five steps, with a 60 second stress relaxation between each step. The Young's modulus was calculated from the slope of the resultant stress versus strain chart for each sample. Four separate samples were prepared and analyzed for each condition. For stress relaxation studies, hydrogels were compressed to 15% strain and maintained while the stress was measured as a function of time.

### Cell Culture and Seeding on HA Hydrogels

Primary lung cancer cells were obtained from patient lung tumor tissues collected upon resection (lobectomy) following patient consent (Ottawa Hospital Research Ethics Board; Protocol # 20120559-01H). Areas containing tumor were identified by routine gross pathological examinations as: well differentiated neuroendocrine tumor, solid predominant adenocarcinoma or poorly differentiated squamous cell carcinoma. Cells were dissociated using collagenase and cultured on 6-well plates in 10% FBS in RPMI-1640 for 7 passages. 4000 cells in 60 μL of 1% FBS in RPMI-1640 was added to each well and cells were cultured on gels for 3 days prior to fixation with 49% PFA.

Commercial lung cancer cells (NCI-H446 and NCI-H1299) were cultured in 10% FBS in RPMI-1640 media. 4000 cells in 60 μL of 1% FBS in RPMI-1640 media was added to each well and cells were cultured on gels for 5 days prior to fixation with 4% PFA.

SMCs were cultured according to reported protocols.^{[12a]} In brief, cells were cultured in M231 media with Smooth Muscle Growth Supplement (SMGS) and gentamycin (ThermoFisher Scientific) at 37° C. with 5% CO₂. Cells were passaged using 0.05% trypsin/EDTA for 3 min at 37° C., and inhibited using M231 media. 3000 cells in 45 μL of M231 without SMGS were added to each well of a 384-well plate containing 3D HA hydrogels.

### Gelatin Zymography

After 1 day of culture, conditioned media from 384-well plates was collected from each well for gelatin zymography. 8% polyacrylamide (PA) gels were used, and were prepared as previously described^{[22]} In general, the separating gel component of the zymography gels comprised 7.5 mL of 1.5 M Tris buffer (pH 8.8), 8.0 mL polyacrylamide solution (30%), 10.2 mL gelatin A (3 mg/mL), 2.7 mL water, 300 μL SDS, 300 μL 10% ammonium persulfate, and 36 μL TEMED. The separating gel was allowed to gel for 40 min at room temperature. The stacking gel was then prepared by mixing 5.0 mL of 0.5 M Tris buffer (pH 6.8), 2.6 mL polyacrylamide solution (30%), 12.2 mL water, 200 μL SDS, 150 μL 10% ammonium persulfate, and 30 μL TEMED. The stacking gel was poured on top of each gel and allowed to sit for 40 mins.

For each sample, 12 μL of conditioned media was mixed with 3 μL loading dye, and 12 μL was loaded into each lane. Running buffer comprising 25 mM Tris, 190 mM glycine and 0.1% SDS was used for gel electrophoresis. Following protein separation on the PA gels, gels were washed in 2.5% Triton X (3 times, 10 min each) to remove residual SDS, followed by DI water (5 times, 30 min each). Gels were then placed in an incubation buffer (50 mM Tris buffer pH 7.5 containing 5 mM CaCl₂, 200 mM NaCl) and incubated at 37° C. overnight. The next day, zymography gels were stained with 0.4% Coumassie Blue for 2 h, followed by destaining in a mixture of 2.5:10:50 (acetic acid: methanol: water) until the white bands were visible.

### Immunocytochemistry

To fix cells cultured in HA hydrogels, media was first carefully removed using a pipette, and 50 μL of 4% PFA (dissolved in PBS) was added for 1.5 h at room temperature. Gels were then carefully washed with 75 μL PBS (4 times, 20 min each with gentle agitation), and 1% BSA in PBS containing 0.1% TritonX was used to block non-specific interactions for at least 1 h.

Primary antibodies (1/100 dilution for each antibody) were diluted in 1% BSA/PBS containing 0.1% TritonX and 20 μL were added to each well, and incubated at 4° C. overnight with gentle agitation. The following primary antibodies were used: mouse anti-CD44 [F10-44-2] (ab6124, Abcam), rabbit anti-CD44v6 exon v6 (AB2080, Millipore), rabbit anti-integrin alpha 5 [EPR7854] (ab150361, Abcam), rabbit anti-integrin alpha V [EPR16800] (ab179475, Abcam), rabbit anti-integrin beta 1 (ab183666, Abcam), rabbit anti-integrin beta 3 (ab197662, Abcam), rabbit anti-integrin beta 5 (ab15459, Abcam). Gels were then washed extensively with 1% BSA/PBS containing 0.1% TritonX with gentle agitation (5 times, at least 1 h each time).

Secondary antibodies (goat anti-mouse-AlexaFluor488, donkey anti-mouse AlexaFluor555 or goat anti-rabbit-AlexaFluor 488, 1/100 dilution) and Hoechst (1/1000 dilutions) were diluted into the same solution in 1% BSA/PBS containing 0.1% TritonX and filtered through a 0.2 μm syringe filter. 20 μL was added into each well, and incubated at room temperature for 3 h. Gels were then washed using 1% BSA/PBS containing 0.1% TritonX extensively (5 times, at least 1 h each time with gentle agitation). Cells in the gels were then imaged in the 384-well plates.

### Image Acquisition

Images were acquired using either an Olympus Fluoview (FV1000) inverted confocal microscope (for immunocytochemistry staining) or a Cellomics (VTI) high content imaging instrument equipped with a brightfield and an inverted confocal microscope (for quantification of cell invasion and viability). The same microscope settings were used for each set of analyses comprising multiple cell types. Negative controls were performed using samples stained with secondary antibodies only (i.e. without primary antibodies). Samples that contained multiple wavelengths were imaged using sequential irradiation of multiple lasers to prevent fluorescent crosstalk. 10× and 20× magnification (5-10 μm step size between Z-stacks) were used for immunocytochemistry staining (Olympus Fluoview) and 5× magnification (30 μm step size between Z-stacks) was used for quantification of cell invasion (Cellomics).

### Quantification of Cell Invasion and Cell Viability

After 5 days of culture in 3D HA gels, 30 μL media was carefully removed. To each well, 20 μL of a solution containing SyTox Green (1/50,000) in PBS was added and incubated for 1.5 h at 37° C. Gels were then carefully washed with PBS twice. Cells were then fixed with 4% PFA for 1.5 h in the dark at room temperature. PFA was removed and gels were washed with PBS. A solution of Hoechst (1/1000, 20 μL) was then added to each well for at least 1.5 h in the dark, followed by washing with PBS (3 times). To label the gel surface, fluorescent microspheres (100 nm, orange, Phosphorex) were diluted in PBS (1/100) and centrifuged to separate larger, aggregated microspheres. 20 μL of the resulting supernatant were then added to each well and the 384-well plate was left undisturbed overnight. For drug screening experiments involving treatment with 80 drugs simultaneously, silica gel particles (40-63 μm, 230-400 mesh, SiliCycle) were used to label the cell surface. A slurry solution (5 mg/mL) of silica gel particles in PBS containing 0.01% sodium azide was mixed and immediately added to each well. Particles settled after 20 seconds and plates could be immediately imaged.

Z-stacked images were obtained using a Cellomics high content imaging instrument as described above. An algorithm was developed on ImageJ to quantify cell invasion and viability. Briefly, using the nuclear stain (Hoechst) channel, the three-dimensional Z-stack image is compressed into a 2D XY projection to identify the two-dimensional (X,Y) position of the cells. For each of these XY coordinates identified as cells, signal intensity peaks along the Z-axis corresponds to the Z-position of a cell, and the maximum peak intensity along that same Z-axis in the second channel (560 nm confocal laser for fluorescent beads or bright field for silica gel particles) corresponds to the top of the gel.

The difference between the Z-positions of the two channels (cell nuclei and fluorescent beads/silica gel particles) is equal to the distance of cell invasion. The signal intensity from the third channel (SyToxGreen) at each cell's three-dimensional position (X, Y, Z) is used to determine the cell viability. Viability is represented as SyToxGreen-negative cells (live cells).

### Quantitative PCR Analysis

RNA was isolated using Trizol (Life Technologies). At least four wells in a 384-well plate were used for each condition. 8000 cells were seeded into each well and allowed to grow for 5 days prior to RNA isolation. Media was carefully removed from each well, 50 μL cold Trizol was added to each well, and the mixture was mixed several times using a pipette with a wide-orifice tip. The contents of at least four wells were pooled together for each condition, and vortexed until the gel was dissociated. RNA was extracted using manufacturer's protocol using 200 μL chloroform for for 1 mL trizol lysate. RNA was purified using a RNA Clean-up Kit (Macherey-Nagel NucleoSpin, Ref#: 740955.250) according to manufacturer's protocol. RNA was reverse transcribed into cDNA normalizing to 100 ng of template.

The primers were designed to span the exon-exon junction to prevent genomic DNA amplification. The primer pairs used were as follows: TSC2 FP ATGTGGTTCATCAGGTGCCG, TSC2 RP ACGTCTGTATCCTCTTGGGTC, gapdh FP AGGTCGGTGTGAACGGATTTG, gapdh RP TGTAGACCATGTAGTTGAGGTCA. Quantification was performed using Pfaffl's ΔΔCt method and GAPDH was used as the housekeeping gene.

Efficiency of the primers was derived by a relative standard curve using a positive template. Primer efficiencies were 1.932 (GAPDH) and 2.023 (TSC2).

### Statistical Analysis

All statistical analyses were performed using GraphPad Prism version 6.00 for Mac (GraphPad Software, San Diego, Calif., USA). Differences amongst two treatments were assessed using an unpaired two-tailed t-test, while differences among groups of three or more treatments were assessed by one-way or two-way ANOVA with Tukey post hoc tests to identify statistical differences, unless otherwise stated in the figure captions. An α level of 0.05 was set as the criterion for statistical significance. Graphs are annotated with p values represented as *p≤0.05, **p≤0.01, ***p≤0.001, ****p≤0.0001. All data are presented as mean+standard deviation.

### Development of a 3D Hydrogel Model of Glioblastoma (GBM) Invasion

Hydrogels are prepared by functionalizing relevant ECM molecules in the GBM microenvironment, namely hyaluronan, methylcellulose functionalized with thiols (MC-SH), adhesive peptides and enzymatically degradable peptides, with mutually reactive Diels-Alder functional groups. These are then combined to form a hydrogel comprising relevant tumour-mimetic ECM components. The final hydrogel formulation comprised 1 weight % methylfuran functionalized hyaluronan (HA-mF 242 kDa, 60% substitution), 2.3 mM bis-maleimide functionalized MMP-degradable peptide crosslinker (Mal-KKGGPQGIWGQKGPQGIWGQGK(Mal)S), 400 μM maleimide-functionalized fibronectin-mimetic peptide (Mal-SKAGPHSRNRGDSPG), and 0.1 mg/mL MC-SH. The hydrogels are washed once with PBS and thrice with cell culture media to remove unreacted components, and equilibrate the gels with the media.

Patient-derived GBM cell lines and healthy human fetal neural stem cells (HFNSC's, a negative control) are then cultured on top of the hydrogels. Cells were then treated with aDT-ITGB1 conjugates at a concentration of approximately 50 nM. Following this culture period, cells are fixed with 4% paraformaldehyde (PFA) and stained with Hoechst and phalloidin to visualize cell position and shape, or live/dead stains for cell viability readouts. The tops of the hydrogels are labelled with fluorescent beads and the cell-laden hydrogels are imaged using 3D z-stack confocal microscopy. A custom algorithm is used to analyse the relative depth of the cells compared to a threshold volume beneath the surface of the gels to quantify cell invasion.

One human fetal neural stem cell line and two patient derived GBM lines were cultured on the gels. The two patient derived cell lines tested invaded into the hydrogels, while the human fetal line grew as a non-invading monolayer on the hydrogel (FIG. 5). Quantitative analysis of these images demonstrated a significant increase in percentage of cells invading and invasion depth for the patient derived lines compared to the HFNSC control.

In summary, the modular design of the hydrogel system according to the present disclosure enables its physicochemical properties to be readily tuned to model disease and tissue-specific ECMs by independently modifying its biochemical composition, matrix stiffness and viscoelasticity. The present inventors hypothesize that this will allow other metastatic diseases involving tissue remodelling and invasion by protease-dependent and -independent mechanisms to be emulated. We demonstrate the breadth of our hydrogel platform to study LAM and lung cancer by culturing both primary human lung cancer cells and commercially-available lung cancer cells. These cancer cells invade into our hydrogels whereas healthy human bronchial epithelial cells do not, highlighting the application of our hydrogel platform to other diseases in addition to LAM.

The 3D hydrogel platform of the present disclosure, using the multi-well plate format, such as 6, 24, 48, 96, 384 or 1536 plates, with automated image acquisition and data analysis, can be readily scaled up using chemical synthesis and used by liquid handling automation to perform larger drug screens. With the ability to monitor invasion and viability at the individual cell level, more detailed analyses of cellular responses to drug treatments are possible, allowing for greater predictive capacity for efficacy than current strategies in drug discovery of anti-metastatic therapeutics.

To summarize, the present disclosure provides an extracellular biomimetic for culturing diseased cells, comprising:

hydrogel matrix,

a first extracellular matrix protein-mimetic peptide crosslinked to the hydrogel matrix, said first extracellular matrix protein-mimetic peptide being responsive to a first substance released by diseased cells upon invasion into the extracellular biomimetic, and

at least one modulating agent enabling cell invasion independent from said first substance.

In an embodiment, the hydrogel matrix comprises hyaluronate or hyaluronic acid, modified with furanyl functional groups.

In an embodiment, the furanyl functional groups are furan, or furan substituted with alkyl-, aryl-, or electron-donating functional groups.

In an embodiment, the modulating agent is at least one viscoelastic component forming reversible crosslinks within the hydrogel matrix.

In an embodiment, the viscoelastic component any one of comprises methyl cellulose, alginate crosslinked with calcium cations, amphiphilic block polymers, amphiphilic block polypeptides, coiled-coil peptides, reconstituted basement membrane protein extract, laminin, or collagen.

In an embodiment, the viscoelastic polymer is methyl cellulose having any one of aldehyde, ketone, hydrazine and thiol functional groups.

In an embodiment, the first extracellular matrix protein-mimetic peptide is further immobilized to the viscoelastic polymer.

In an embodiment, the extracellular biomimetic further comprises a second extracellular matrix protein-mimetic peptide immobilized to the hydrogel matrix and/or the viscoelastic polymer.

In an embodiment, the second extracellular matrix protein-mimetic peptide is present in the amount of less than about 1000 μM.

In an embodiment, the second extracellular matrix protein-mimetic peptide is present in the amount of about 25 μM to about 250 μM.

In an embodiment, the second extracellular matrix protein-mimetic peptide is any one or combination of vitronectin-mimetic peptide and fibronectin-mimetic peptide.

In an embodiment, the first substance released by diseased cells is an enzyme. In an embodiment, the enzyme is matrix metalloproteinase (MMP). In an embodiment, the first extracellular matrix protein-mimetic peptide is maleimide-modified collagen I-derived peptide crosslinker degradable by the MMP.

In an embodiment, the present disclosure also provides a cell culture kit comprising the extracellular biomimetic as disclosed above.

In an embodiment of the cell culture kit the diseased cells are from any invading cells, such as any one of the lung, brain, breast, prostate, skin, liver, colon, pancreas, thyroid, bone, muscle, human pluripotent stem cells and their subsequently differentiated cells.

In an embodiment of the cell culture kit, the diseased cells comprise cells isolated from lung cancer patients or derived from human pluripotent stem cells (hiPSCs) to model lymphangioleiomyomatosis (LAM).

In an embodiment of the cell culture kit,diseased cells comprise hiPSC-derived smooth muscle cells (SMCs) that model lymphangioleiomyomatosis (LAM-SMCs).

In an embodiment of the cell culture kit, the diseased cells comprise cells treated with one or any combination of inhibitors selected from the group consisting of those that inhibit:
ABL1
ADENOSINE DEAMINASE
AKT3
ALK
ANDROGEN
AROMATASE
AURORA KINASE
BCL-2
BRAF
BRD
BTK
CALCINEURIN
CCR5
CDK
CXCR
CYTOCHROME P450
DAGK
DNA METHYLTRANSFERASE
DNA TOPOISOMERASE
EGFR
EPH
ERK
Fibroblast Growth Factor Receptors
FARNESYLTRANSFERASE
FLT
FRAP
GS K3
HDAC
HEAT SHOCK PROTEIN
HEDGEHOG
IRE1
ITGB1
JAK2
KDR
KINESIN-LIKE SPINDLE PROTEIN
KIT
LCK
LIMK1
LYN
MAP2K
MDM2
P38B
P70S6K
PARP
PDGFR
PI3K
PKC
PLK1
PIM2
PROTEASOME
RAF1
Rho-associated protein kinase
RET
Src
SIRT2
SPHINGOSINE KINASE
TANKYRASE
TUBULIN
WNT,
   or one or any combination of agonists selected from the group consisting of:
GLUCOCORTICOID
PKM2
PROGESTERONE
RXR
S1P RECEPTOR.

In an embodiment of the cell culture kit, the kit includes 6, 24, 48, 96, 384 or 1536 well plates.

In an embodiment the present disclosure provides a drug screening method comprising:
culturing diseased cells in the extracellular biomimetic as disclosed above;
quantifying invasion and viability of the diseased cells;
administering candidate drug compounds to the biomimetic; and
identifying compounds that reduce both the invasion and viability of the diseased cells.

In an embodiment of the method the quantifying step comprises measuring the invasion of the diseased cells by staining cells with fluorescent dyes, automated confocal imaging, and automated analysis by an image analysis software program such as custom Image J macros.

In an embodiment of the method the quantifying step comprises measuring the viability of the diseased cells by staining the dead cells with fluorescent dyes, automated microscopic imaging such as confocal imaging, and automated analysis by an image analysis software program such as custom Image J macros.

In an embodiment of the method, the diseased cells are from any one of the lung, brain, skin, breast, prostate, liver, colon, pancreas, thyroid, bone, muscle, human pluripotent stem cells and their subsequently differentiated cells.

In an embodiment of the method the diseased cells comprise cells isolated from lung cancer patients or derived from human pluripotent stem cells (hiPSCs) to model lymphangioleiomyomatosis (LAM).

In an embodiment of the method the diseased cells comprise hiPSC-derived smooth muscle cells (SMCs) that model lymphangioleiomyomatosis (LAM-SMCs).

In an embodiment of the method the diseased cells comprise cells treated with one or any combination of inhibitors selected from the group consisting of those that inhibit:

ABL1
ADENOSINE DEAMINASE
AKT3
ALK
ANDROGEN
AROMATASE
AURORA KINASE
BCL-2
BRAF
BRD
BTK
CALCINEURIN
CCR5
CDK
CXCR
CYTOCHROME P450
DAGK
DNA METHYLTRANSFERASE
DNA TOPOISOMERASE
EGFR
EPH
ERK
Fibroblast Growth Factor Receptors
FARNESYLTRANSFERASE
FLT
FRAP
GSK3
HDAC
HEAT SHOCK PROTEIN
HEDGEHOG
IRE1
ITGB1
JAK2
KDR
KINESIN-LIKE SPINDLE PROTEIN
KIT
LCK
LIMK1
LYN
MAP2K
MDM2
P38B
P70S6K
PARP
PDGFR
PI3K
PKC
PLK1
PIM2
PROTEASOME
RAF1
RET
Rho-associated protein kinase
Src
SIRT2
SPHINGOSINE KINASE
TANKYRASE
TUBULIN
WNT,
   or one or any combination of agonists selected from the group consisting of:
GLUCOCORTICOID
PKM2
PROGESTERONE
RXR
S1P RECEPTOR.

In an embodiment the method is carried out in a cell culture kit having 6, 24, 48, 96, 384 or 1536 well plates. In an embodiment each plate of the cell culture kit contains both diseased cells and control cells.

### REFERENCES p0 [1] a) F. Sabeh, R. Shimizu-Hirota, S. J. Weiss, J. Cell Biol. 2009, 185, 11; b) P. Friedl, K. Wolf, Nat. Rev. Cancer 2003, 3, 362.

[2] J. F. de Groot, G. Fuller, A. J. Kumar, Y. Piao, K. Eterovic, Y. Ji, C. A. Conrad, Neuro-Oncology 2010, 12, 233.
[3] a) M. J. Bissell, D. C. Radisky, A. Rizki, V. M. Weaver, O. W. Petersen, Differentiation 2002, 70, 537; b) C. Yang, M. W. Tibbitt, L. Basta, K. S. Anseth, Nat. Mater. 2014, 13, 645; c) O. Chaudhuri, L. Gu, M. Darnell, D. Klumpers, S. A. Bencherif, J. C. Weaver, N. Huebsch, D. J. Mooney, Nat. Commun. 2015, 6, 6364.
[4] A. Glentis, P. Oertle, P. Mariani, A. Chikina, F. El Marjou, Y. Attieh, F. Zaccarini, M. Lae, D. Loew, F. Dingli, P. Sirven, M. Schoumacher, B. G. Gurchenkov, M. Plodinec, D. M. Vignjevic, Nat. Commun. 2017, 8, 924.
[5] O. W. Petersen, L. Ronnov-Jessen, A. R. Howlett, M. J. Bissell, Proc. Natl. Acad. Sci. U. S. A. 1992, 89, 9064.
[6] S. R. Caliari, J. A. Burdick, Nat. Methods 2016, 13, 405.
[7] a) S. A. Fisher, R. Y. Tam, A. Fokina, M. M. Mahmoodi, M. D. Distefano, M. S. Shoichet, Biomaterials 2018, 178, 751; b) J. Patterson, J. A. Hubbell, Biomaterials 2010, 31, 7836.
[8] M. Ehrbar, A. Sala, P. Lienemann, A. Ranga, K. Mosiewicz, A. Bittermann, S. C. Rizzi, F. E. Weber, M. P. Lutolf, Biophys. J. 2011, 100, 284.
[9] A. Winer, S. Adams, P. Mignatti, Mol. Cancer Ther. 2018, 17, 1147.
[10] a) H. A. Kenny, M. Lal-Nag, E. A. White, M. Shen, C. Y. Chiang, A. K. Mitra, Y. Zhang, M. Curtis, E. M. Schryver, S. Bettis, A. Jadhav, M. B. Boxer, Z. Li, M. Ferrer, E. Lengyel, Nat. Commun. 2015, 6, 6220; b) N. A. Evensen, J. Li, J. Yang, X. Yu, N. S. Sampson, S. Zucker, J. Cao, Plos One 2013, 8, e82811; c) Y. Fang, R. M. Eglen, SLAS Discovery 2017, 22, 456.
[11] a) T. N. Darling, G. Pacheco-Rodriguez, A. Gorio, E. Lesma, C. Walker, J. Moss, Lymphatic Res. Biol. 2010, 8, 59; b) E. P. Henske, F. X. McCormack, J. Clin. Invest. 2012, 122, 3807.
[12] a) L. M. Julian, S. P. Delaney, Y. Wang, A. A. Goldberg, C. Dore, J. Yockell-Lelievre, R. Y. Tam, K. Giannikou, F. McMurray, M. S. Shoichet, M. E. Harper, E. P. Henske, D. J. Kwiatkowski, T. N. Darling, J. Moss, A. S. Kristof, W. L. Stanford, Cancer Res. 2017, 77, 5491; b) S. P. Delaney, L. M. Julian, A. Pietrobon, J. Yockell-Lelièvre, C. Doré, T. T. Wang, V. C. Doyon, A. Raymond, D. A. Patten, M.-E. Harper, H. Sun, W. L. Stanford, BioRxiv. 2019, doi: https://doi.org/10.1101/683359
[13] C. J. Whatcott, H. Han, R. G. Posner, G. Hostetter, D. D. Von Hoff, Cancer Discov 2011, 1, 291.
[14] G. Pacheco-Rodriguez, W. K. Steagall, D. M. Crooks, L. A. Stevens, H. Hashimoto, S. Li, J. A. Wang, T. N. Darling, J. Moss, Cancer Res. 2007, 67, 10573.
[15] S. Misra, V. C. Hascall, R. R. Markwald, S. Ghatak, Front. Immunol. 2015, 6, 201.
[16] S. C. Owen, S. A. Fisher, R. Y. Tam, C. M. Nimmo, M. S. Shoichet, Langmuir 2013, 29, 7393.
[17] M. K. Glassberg, S. J. Elliot, J. Fritz, P. Catanuto, M. Potier, R. Donahue, W. Stetler-Stevenson, M. Karl, J. Clin. Endocrinol. Metab. 2008, 93, 1625.
[18] B. Suki, J. H. Bates, Respir. Physiol. Neurobiol. 2008, 163, 33.
[19] A. J. Booth, R. Hadley, A. M. Cornett, A. A. Dreffs, S. A. Matthes, J. L. Tsui, K. Weiss, J. C. Horowitz, V. F. Fiore, T. H. Barker, B. B. Moore, F. J. Martinez, L. E. Niklason, E. S. White, Am. J. Respir. Crit. Care Med. 2012, 186, 866.
[20] M. Y. Hsu, D. T. Shih, F. E. Meier, P. Van Belle, J. Y. Hsu, D. E. Elder, C. A. Buck, M. Herlyn, Am. J. Pathol. 1998, 153, 1435.
[21] S. Suzuki, A. Oldberg, E. G. Hayman, M. D. Pierschbacher, E. Ruoslahti, EMBO J. 1985, 4, 2519.
[22] X. Hu, C. Beeton, J. Vis. Exp. 2010, 45, 2445.
[23] A. R. Cameron, J. E. Frith, G. A. Gomez, A. S. Yap, J. J. Cooper-White, Biomaterials 2014, 35, 1857.
[24] a) K. C. Williams, M. G. Coppolino, J. Cell Sci. 2014, 127, 1712; b) C. Albiges-Rizo, O. Destaing, B. Fourcade, E. Planus, M. R. Block, J. Cell Sci. 2009, 122, 3037.
[25] D. Clements, A. Dongre, V. P. Krymskaya, S. R. Johnson, Plos One 2015, 10, e0126025.
[26] a) C. Underhill, J. Cell Sci. 1992, 103, 293; b) K. H. Heider, H. Kuthan, G. Stehle, G. Munzert, Cancer lmmunol. Immunother. 2004, 53, 567.
[27] C. Li, P. S. Lee, Y. Sun, X. Gu, E. Zhang, Y. Guo, C. L. Wu, N. Auricchio, C. Priolo, J. Li, A. Csibi, A. Parkhitko, T. Morrison, A. Planaguma, S. Kazani, E. Israel, K. F. Xu, E. P. Henske, J. Blenis, B. D. Levy, D. Kwiatkowski, J. J. Yu, J. Exp. Med. 2014, 211, 15.
[28] F. X. McCormack, Y. Inoue, J. Moss, L. G. Singer, C. Strange, K. Nakata, A. F. Barker, J. T. Chapman, M. L. Brantly, J. M. Stocks, K. K. Brown, J. P. Lynch, 3rd, H. J. Goldberg, L. R. Young, B. W. Kinder, G. P. Downey, E. J. Sullivan, T. V. Colby, R. T. McKay, M. M. Cohen, L. Korbee, A. M. Taveira-DaSilva, H. S. Lee, J. P. Krischer, B. C. Trapnell, C. National Institutes of Health Rare Lung Diseases, M. T. Group, N. Engl. J. Med. 2011, 364, 1595.
[29] J. D. Humphries, A. Byron, M. J. Humphries, J. Cell Sci. 2006, 119, 3901.
[30] K. Koshizuka, N. Kikkawa, T. Hanazawa, Y. Yamada, A. Okato, T. Arai, K. Katada, Y. Okamoto, N. Seki, Oncotarget 2017, 9, 3663.

1. An extracellular biomimetic for culturing diseased cells, comprising:
hydrogel matrix,
a first extracellular matrix protein-mimetic peptide crosslinked to the hydrogel matrix, said first extracellular matrix protein-mimetic peptide being responsive to a first substance released by diseased cells upon invasion into the extracellular biomimetic, and
at least one modulating agent enabling cell invasion independent from said first substance.

2. The extracellular biomimetic according to claim 1, wherein the hydrogel matrix comprises hyaluronate or hyaluronic acid, modified with furanyl functional groups.

3. The extracellular biomimetic according to claim 2, wherein the furanyl functional groups are furan, or furan substituted with alkyl-, aryl-, or electron-donating functional groups.

4. The extracellular biomimetic according to claim 1, wherein the modulating agent is at least one viscoelastic component forming reversible crosslinks within the hydrogel matrix.

5. The extracellular biomimetic according to claim 4, wherein the component comprises any one of methyl cellulose, alginate crosslinked with calcium cations, amphiphilic block polymers, amphiphilic block polypeptides, coiled-coil peptides, reconstituted basement membrane protein extract, laminin, or collagen, said methyl cellulose optionally having any one of aldehyde, and thiol functional groups.

6. (canceled)

7. The extracellular biomimetic according to claim 4, wherein the first extracellular matrix protein-mimetic peptide is further immobilized to the viscoelastic polymer.

8. The extracellular biomimetic according to claim 4, further comprising a second extracellular matrix protein-mimetic peptide immobilized to the hydrogel matrix and/or the viscoelastic polymer.

9. The extracellular biomimetic according to claim 8 wherein the second extracellular matrix protein-mimetic peptide is present in the amount of about 25 μM to 1000 μM.

10. (canceled)

11. The extracellular biomimetic according to claim 8, wherein the second extracellular matrix protein-mimetic peptide is any one or combination of vitronectin-mimetic peptide and fibronectin-mimetic peptide.

12. The extracellular biomimetic according to claim 1, wherein the first substance released by diseased cells is an enzyme.

13. The extracellular biomimetic according to claim 12, wherein the enzyme is matrix metalloproteinase (MMP).

14. The extracellular biomimetic according to claim 13, wherein the first extracellular matrix protein-mimetic peptide is maleimide-modified collagen I-derived peptide crosslinker degradable by the MMP.

15. A cell culture kit comprising the extracellular biomimetic according to claim 1 and cells.

16. The cell culture kit according to claim 15 wherein the cells are diseased cells from any invading cells, such as any one of the lung, brain, breast, prostate, skin, liver, colon, pancreas, thyroid, bone, muscle, human pluripotent stem cells and their subsequently differentiated cells.

17. The cell culture kit according to claim 15 wherein the cells are diseased cells isolated from lung cancer patients or derived from human pluripotent stem cells (hiPSCs) to model lymphangioleiomyomatosis (LAM), or derived from hiPSC smooth muscle cells (SMCs) that model lymphangioleiomyomatosis (LAM-SMCs).

18. (canceled)

19. The cell culture kit according to claim 15, wherein the cells are diseased cells treated with one or any combination of inhibitors selected from the group consisting of those that inhibit:
ABL1
ADENOSINE DEAMINASE
AKT3
ALK
ANDROGEN
AROMATASE
AURORA KINASE
BCL-2
BRAF
BRD
BTK
CALCINEURIN
CCR5
CDK
CXCR
CYTOCHROME P450
DAGK
DNA METHYLTRANSFERASE
DNA TOPOISOMERASE
EGFR
EPH
ERK
Fibroblast Growth Factor Receptors
FARNESYLTRANSFERASE
FLT
FRAP
GSK3
HDAC
HEAT SHOCK PROTEIN
HEDGEHOG
IRE1
ITGB1
JAK2
KDR
KINESIN-LIKE SPINDLE PROTEIN
KIT
LCK
LIMK1
LYN
MAP2K
MDM2
P38B
P70S6K
PARP
PDGFR
PI3K
PKC
PLK1
PIM2
PROTEASOME
RAF1
Rho-associated protein kinase
RET
Src
SIRT2
SPHINGOSINE KINASE
TANKYRASE
TUBULIN
WNT,
or one or any combination of agonists selected from the group consisting of:
GLUCOCORTICOID
PKM2
PROGESTERONE
RXR
S1P RECEPTOR.

20. The cell culture kit according to claim 15, having 6, 24, 48, 96, 384 or 1536 well plates.

21. A drug screening method comprising:
culturing diseased cells in the extracellular biomimetic according to any one of claim 1;
quantifying invasion and viability of the diseased cells;
administering candidate drug compounds to the biomimetic; and
identifying compounds that reduce both the invasion and viability of the diseased cells.

22. The method according to claim 21, wherein the quantifying step comprises measuring the invasion of the diseased cells by staining cells with fluorescent dyes, automated confocal imaging, and automated analysis by an image analysis software program such as custom Image J macros.

23. The method according to claim 21, wherein the quantifying step comprises measuring the viability of the diseased cells by staining the dead cells with fluorescent dyes, automated microscopic imaging such as confocal imaging, and automated analysis by an image analysis software program such as custom Image J macros.

24-29. (canceled)
